(19) United States
(12) Patent Application Publication
(10) Pub. No.: US 2015/0005265 A1
(43) Pub. Date: Jan. 1, 2015

(54) METHODS AND COMPOSITIONS FOR COMBINATION THERAPY USING P13K/MTOR INHIBITORES

(71) Applicant: Susan Stewart, Lynnfield, MA (US)

(72) Inventor: Susan Stewart, Lynnfield, MA (US)

(21) Appl. No.: 14/366,662

(22) PCT Filed: Dec. 21, 2012
(86) PCT No.: PCT/US2012/071485

§ 371 Date: Jun. 18, 2014

(51) Int. Cl. A61K 31/58; A61K 31/5377; A61K 31/519; A61K 31/635; A61K 31/506; A61K 31/436; A61K 31/4745

(52) U.S. Cl. CPC A61K 31/58; A61K 31/436; A61K 31/5377; A61K 31/4745; A61K 31/635; A61K 31/506; A61K 31/519
USPC 514/157; 514/171

(57) ABSTRACT

The present invention provides methods and compositions for treating cancers using a combination of a CYP 1 7 inhibitor and an additional therapeutic agent which modulates the PBK/Akt/mTOR pathway. In one aspect, the invention provides methods for the treatment of a disorder in a human subject. In some embodiments, the disorder is a neoplastic disorder. In some embodiments, the neoplastic disorder is a cancer. In some embodiments, the method comprises administering to said subject a 17a-hydroxylase/C17,20-lyase inhibitor (CYP17 inhibitor) and an additional agent, wherein the additional agent is a PBK inhibitor and/or mTOR inhibitor.

### CROSS-REFERENCE

This application claims the benefit of U.S. Provisional Application No. 61/579,452, filed Dec. 22, 2011, which application is hereby incorporated by reference in its entirety.

### BACKGROUND OF THE INVENTION

Neoplastic and hyperproliferative disorders represent an area of particular therapeutic interest. In recent years, cancer diagnoses have continued to increase, with cancers that are hormone-dependent, namely androgen-related prostate cancer in men and estrogen-related breast cancer in women, meriting special note.

Prostate cancer is currently the second leading cause of cancer-related deaths in men after lung cancer, and second in prevalence only to skin cancer. The primary course of treatment for patients diagnosed with organ-confined prostate cancer is usually prostatectomy or radiotherapy. Not only are these treatments highly invasive and have undesirable side effects, such localized treatments are ineffective on metastatic prostate cancer, and a large percent of individuals who receive these localized treatments will suffer from recurring cancer that is non-localized and resistant to hormone therapy.

In the United States, breast cancer incidence in women has increased from one out of every twenty women in 1960 to one out of every eight women in 2005, and it is the most common cancer among white and African-American women. Most options for women diagnosed with breast cancer, i.e., surgery, radiation and chemotherapy, are also highly invasive and have significant side effects.

Hormone therapy is another treatment option for individuals diagnosed with prostate or breast cancer. Hormone therapy is a form of systemic treatment for prostate or breast cancer, wherein hormone ablation agents are used to suppress the production or block the effects of hormones, such as estrogen and progesterone, which are believed to promote the growth of breast cancer, as well as testosterone and dihydrotestosterone, which are believed to promote the growth of prostate cancer. This therapy is less invasive than surgery and does not have many of the side effects associated with chemotherapy or radiation. In addition, hormone therapy may be used by itself or in addition to localized therapy, and has been shown to be effective in individuals with metastatic neoplasia.

While hormone therapy is less invasive and may be used on more advanced stages of cancer, some individuals administered current hormone therapy treatments may not respond completely, or even partially, to such treatments. Current hormone therapy treatments may offer temporal remission of cancer, but these treated cancers can relapse or recur, and upon recurrence, these cancers often have developed a resistance to hormonal therapy. Due to the typically aggressive nature of these recurrent cancers, and their resistance to hormonal therapy, patients with these conditions are often left with few options for treatment.

Despite the progress made in the treatment of cancer, there remains a need for more effective ways to treat cancer, such as, but not limited to, prostate cancer and breast cancer. Additionally, there is a need for effective anti-cancer treatment options for patients who are not responding to current anti-cancer treatments, as well as for effective anti-cancer treatment options for patients whose cancers have recurred.

### SUMMARY OF THE INVENTION

In one aspect, the invention provides methods for the treatment of a disorder in a human subject. In some embodiments, the disorder is a neoplastic disorder. In some embodiments, the neoplastic disorder is a cancer. In some embodiments, the method comprises administering to said subject a 17α-hydroxylase/C17,20-lyase inhibitor (CYP17 inhibitor) and an additional agent, wherein the additional agent is a PI3K inhibitor and/or mTOR inhibitor.

In some embodiments, the CYP17 inhibitor is a 17-heteroarylsteroid compound or a pharmaceutically acceptable salt, analog, N-oxide, prodrug, or solvate thereof. In some embodiments, the 17-heteroarylsteroid compound is Compound (I):

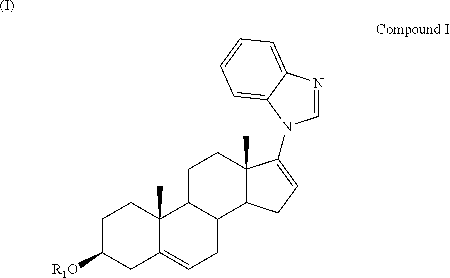

or a pharmaceutically acceptable salt, analog, N-oxide, prodrug, or solvate thereof, wherein: R₁ is H or acetyl.

In a particular embodiment of Compound (I), R₁ is H.

In other embodiments, the 17-heteroarylsteroid compound is abiraterone alcohol or abiraterone acetate, or a pharmaceutically acceptable salt, analog, N-oxide, prodrug, or solvate thereof.

In another aspect, the invention provides a method of treating prostate cancer in a subject in need thereof, comprising administering to said subject a CYP17 inhibitor and at least one additional therapeutic agent, wherein the additional therapeutic agent is a PI3K inhibitor and/or mTOR inhibitor if blood PSA levels in said subject has increased in at least two successive occasions at least one week apart.

In another embodiment, the method comprises administering a CYP17 inhibitor and at least one additional therapeutic agent, wherein the additional therapeutic agent is a PI3K inhibitor and/or mTOR inhibitor, to said subject if blood PSA level is 4 ng/ml or above.

In yet another aspect, the invention provides a method of treating prostate cancer in a subject in need thereof, comprising administering a CYP17 inhibitor and at least one additional therapeutic agent, wherein the additional therapeutic agent is a PI3K inhibitor and/or mTOR inhibitor, to said subject if said subject is determined to harbor a mutation or copy number variation in a gene associated with the PI3K/mTOR pathway.

In some embodiments, said mutation or copy number variation is selected from the group consisting of PTEN mutations, PTEN loss-of-heterozygosity, PIK3CA mutations, PIK3CA amplifications, AKT mutations, AKT amplifications, and P85α mutations.

In some embodiments, the CYP17 inhibitor is Compound I, abiraterone alcohol, abiraterone acetate.

In some embodiments of any of the methods described herein, the mTOR inhibitor directly binds and inhibits mTORC1 and mTORC2.

In other embodiments, the mTOR inhibitor is selectively active against mTORC1 as compared to mTORC2.

In particular embodiments, the mTOR inhibitor is rapamycin, temsirolimus, umirolimus, zotarolimus, or any analogues or derivatives thereof.

In other particular embodiments, the mTOR inhibitor is not everolimus.

In other particular embodiments, the mTOR inhibitor is not rapamycin or a rapamycin analog.

In some embodiments, the mTOR inhibitor is a TOR kinase inhibitor (TOR-KI).

In particular embodiments, the mTOR inhibitor is OSI-027, INK-128, AZD-8055, AZD-2014, Palomid 529, Pp-242, BEZ235, AZD-8055, BGT226, XL765, GDC-0980, GSK2126458, PF-04691502, PF-05212384, or any analogues or derivatives thereof.

In some embodiments, the mTOR inhibitor also inhibits PI3K.

In some embodiments of any of the methods described herein, the additional therapeutic agent is a PI3K inhibitor.

In particular embodiments, the PI3K inhibitor is a pan-PI3K inhibitor.

In other particular embodiments, the PI3K inhibitor selectively inhibits a class I PI3K family member relative to at least one other class I PI3K family member.

In other particular embodiments, the PI3K inhibitor selectively inhibits PI3Kα, PI3Kβ, PI3Kγ, PI3Kδ, or some combination thereof.

In some embodiments, the PI3K inhibitor also inhibits mTOR.

In particular embodiments, the PI3K inhibitor is SF1126, SF1101, BEZ235, BKM120, BYL719, BGT-226, XL-147, GDC-0941, ZSTK-474, PX-866, GDC-0980, PKI-587, PF-04691502, BWT33597, PI-103, CAL-101, GNE-477 or any derivatives thereof.

In some embodiments of any of the methods described herein, the subject is a human.

In some embodiments, the cancer comprises a heterogeneous tumor.

In some embodiments, the cancer is bone cancer, breast cancer, cervical cancer, endometrial cancer, leukemia, lung cancer, lymphoma, ovarian cancer, prostate cancer, skin cancer, or testicular cancer.

In some embodiments, the cancer is prostate cancer or breast cancer.

In some embodiments, the prostate cancer is castration-resistant prostate cancer.

In some embodiments, Compound (I) or abiraterone alcohol or abiraterone acetate and the additional therapeutic agent are administered concurrently to the subject.

In other embodiments, Compound (I) or abiraterone alcohol or abiraterone acetate and the additional therapeutic agent are administered separately to the subject.

In some embodiments, methods described herein comprise administering Compound (I), abiraterone alcohol, or abiraterone acetate and the additional therapeutic agent for a period of about 3 days to about 12 months.

In other embodiments, methods described herein comprise administering Compound (I), abiraterone alcohol, or abiraterone acetate and the additional therapeutic agent for a period of about 28 days to about 3 months.

In particular embodiments, the methods comprise administering Compound (I), abiraterone alcohol, or abiraterone acetate and the additional therapeutic agent for a period of over 45 days.

In still other embodiments, the methods comprise administering Compound (I), abiraterone alcohol, or abiraterone acetate and the additional therapeutic agent for a period of over 60 days.

In yet more particular embodiments, the methods comprise administering Compound (I), abiraterone alcohol, or abiraterone acetate and the additional therapeutic agent for a period of over 90 days.

In some embodiments, methods described herein comprise administering between about 30 to about 175 mg/kg/day of Compound (I), abiraterone alcohol, or abiraterone acetate.

In other embodiments, the methods comprise administering between about 25 mg/kg/day to about 50 mg/kg/day of Compound (I), abiraterone alcohol, or abiraterone acetate.

In yet other embodiments, the methods comprise administering less than 50 mg/kg/day of Compound (I), abiraterone alcohol, or abiraterone acetate.

In some embodiments, methods described herein comprise administering about 325 mg to about 3500 mg of Compound (I), abiraterone alcohol, or abiraterone acetate.

In particular embodiments, the methods comprise administering between 600 mg and 1950 mg of Compound (I), abiraterone alcohol, or abiraterone acetate.

In yet more particular embodiments, the methods comprise administering about 600 mg, about 975 mg, about 1300 mg, or about 1950 mg of Compound (I), abiraterone alcohol, or abiraterone acetate.

In some embodiments, the methods described herein comprise administering between about 0.01 and 10 mg/kg of the additional therapeutic agent.

In particular embodiments, the methods comprise administering between about 0.01 and 1 mg/kg of the additional therapeutic agent.

In other particular embodiments, the methods comprise administering between about 0.1 and 2 mg/kg of the additional therapeutic agent.

In other particular embodiments, the methods comprise administering between about 0.5 and 5 mg/kg of the additional therapeutic agent.

In some embodiments, the methods comprise administering between about 1 and 10 mg/kg of the additional therapeutic agent.

In some embodiments, the cancer tumor volume decreases after the administration of Compound (I), abiraterone alcohol, or abiraterone acetate and the additional therapeutic agent for said period.

In other embodiments, the cancer tumor volume remains stable after the administration of Compound (I), abiraterone alcohol, or abiraterone acetate and the additional therapeutic agent for said period.

In particular embodiments, the cancer remains stable as characterized by RECIST guidelines during administration of Compound (I), abiraterone alcohol, or abiraterone acetate and the additional therapeutic agent for said period.

In some embodiments, the methods described herein comprise administering Compound (I), abiraterone alcohol, or abiraterone acetate and/or the additional therapeutic agent to a subject one, two, three, four, five, six, seven, eight, nine, or ten times per day.

In some embodiments, Compound (I), abiraterone alcohol, or abiraterone acetate and/or the additional therapeutic agent can be administered parenterally, intravenously, intramuscularly, intradermally, subcutaneously, intraperitoneally, orally, buccally, sublingually, mucosally, rectally, transcutaneously, transdermally, ocularly, or by inhalation.

In particular embodiments, Compound (I), abiraterone alcohol, or abiraterone acetate is administered as a tablet, a capsule, a cream, a lotion, an oil, an ointment, a gel, a paste, a powder, a suspension, an emulsion, or a solution.

In some embodiments, Compound (I), abiraterone alcohol, or abiraterone acetate is formulated as a solid dispersion composition.

In particular embodiments, the solid dispersion composition is a spray dried dispersion composition.

In some embodiments, the methods described herein comprise administering a therapeutically effective amount of Compound (I), abiraterone alcohol, or abiraterone acetate.

In some embodiments, the methods described herein comprise administering a therapeutically effective amount of the additional therapeutic agent.

In other embodiments, a sub-therapeutic amount of Compound (I), abiraterone alcohol, or abiraterone acetate is administered.

In some embodiments, a sub-therapeutic amount of the additional therapeutic agent is administered.

In particular embodiments, administration of Compound (I), abiraterone alcohol, or abiraterone acetate and the additional therapeutic agent results in a synergistic effect, wherein the synergistic effect is evidenced by a therapeutic effect of administering both Compound (I) and the additional therapeutic agent to a test subject that is more than the additive effects of administering only Compound (I) to a test subject and administering only the additional therapeutic agent to a test subject.

In some embodiments, the additional therapeutic agent inhibits a PI3K or mTOR complex with a potency of less than 1 μM in an in vitro assay.

In particular embodiments, the additional therapeutic agent inhibits a PI3K or mTOR complex with a potency of less than 500 nM in an in vitro assay.

In yet more particular embodiments, the additional therapeutic agent inhibits a PI3K or mTOR complex with a potency of less than 100 nM in an in vitro assay.

In another aspect, the invention provides compositions for the treatment of cancer in a subject. In some embodiments, the composition comprises a 17α-hydroxylase/C₁₇₋₂₀-lyase (CYP17) inhibitor and at least one additional therapeutic agent, wherein the additional therapeutic agent is a PI3K inhibitor and/or mTOR inhibitor. or an analog, a derivative, a metabolite or a pharmaceutically-acceptable salt thereof.

In other embodiments, the 17α-hydroxylase/C₁₇₋₂₀-lyase inhibitor is a compound of Formula (II):

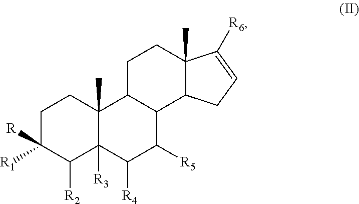

wherein:
either R and R1 are independently H, OH, SH, NH2, N(R7), NHR7, F, OR7, or O(C═O)R7; or R and R1 together form a ketone or an exo-methylene;
a. each occurrence of R₇ is independently H, C₁-C₈-alkyl, arakyl, alkylaryl, alkoxyalkyl, aryl,

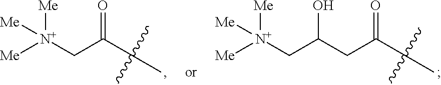 [SMILES: *C(=O)C[N+](C)(C)C ; *C(=O)CC(O)C[N+](C)(C)C]

b. R₂, R₃, R₄, and R₅ are independently H, OH, SH, NH₂, or NHR₇, or together with a neighboring R₂, R₃, R₄, or R₅ form an olefinic bond;
c. R₆ is: a 1-azaazulen-3-yl; 2-alkylindazol-3-yl; pyrazolo-[1,5-a]-pyridin-3-yl; imidazo-[1,2-a]-pyridin-3-yl; pyrazolo-[2,3-a]-pyrimidin-3-yl; pyrazolo-[2,3-c]-pyrimidin-3-yl; imidazo-[1,2-c]-pyrimidin-3-yl; imidazo-[1,2-a]-pyrimidin-3-yl; 4-alkylpyrazolo-[1,5-a]imidazol-3-yl; 2,1-benzoxazol-3-yl; 2,1-benzthiazol-3-yl; imidazo[2,1-b][1,3]oxazol-5-yl; imidazo[2,1-b][1,3]thiazol-5-yl; imidazo-[2,1-b][1,2]isoxazol-6-yl; or 1,2-benzisoxazol-3-yl, group, wherein any of the foregoing groups are optionally-substituted; or a bicyclic structure of Formula III:

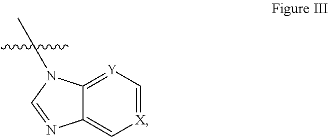

wherein X and Y are independently CH or N, and the bicyclic structure of Formula III is optionally substituted with halogen, chalcogen or C₁-C₄-alkyl; or
wherein R₆ is a bicyclic structure of Formula III wherein one of X and Y is N and the other of X and Y is CH when one or both of R and R₁ are

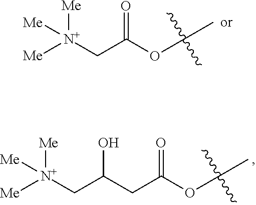 [SMILES: *OC(=O)C[N+](C)(C)C ; *OC(=O)CC(O)C[N+](C)(C)C]

or an analog, a derivative, a metabolite or a pharmaceutically-acceptable salt of any of the foregoing.

In particular embodiments, the compound is Compound I or abiraterone alcohol or abiraterone acetate:

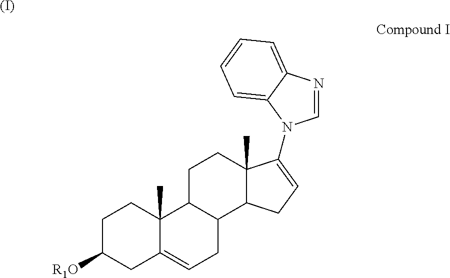

or a pharmaceutically acceptable salt, analog, N-oxide, prodrug, or solvate thereof, wherein: R₁ is H or acetyl.

In some embodiments, the composition comprises about 50 to about 3500 mg of said CYP17 inhibitor.

In some embodiments, the composition comprises about 50 to about 3500 mg of said CYP17 inhibitor and about 5 to about 500 mg of said PI3K inhibitor or mTOR inhibitor.

In some embodiments, the mTOR inhibitor binds to and inhibits both mTORC1 and mTORC2.

In other embodiments, the mTOR inhibitor selectively inhibit mTORC1 as compared to mTORC2.

In particular embodiments, the mTOR inhibitor is rapamycin, temsirolimus, umirolimus, zotarolimus, or any analogues or derivatives thereof.

In other embodiments, the mTOR inhibitor is not everolimus.

In particular embodiments, the mTOR inhibitor is not rapamycin or a rapamycin analog.

In some embodiments, the mTOR inhibitor also inhibits PI3K.

In other particular embodiments, the mTOR inhibitor is a TOR kinase inhibitor (TOR-KI).

In yet more particular embodiments, the mTOR inhibitor is OSI-027, INK-128, AZD-8055, AZD-2014, Palomid 529, Pp-242, BEZ235, AZD-8055, BGT226, XL765, GDC-0980, GSK2126458, PF-04691502, PF-05212384, or any analogues or derivatives thereof.

In some embodiments, the PI3K inhibitor is a pan-PI3K inhibitor.

In particular embodiments, the PI3K inhibitor selectively inhibits a class I PI3K family member relative to at least one other class I PI3K family member.

In other particular embodiments, the PI3K inhibitor selectively inhibits PI3Kα, PI3Kβ, PI3Kγ, PI3Kδ, or some combination thereof.

In some embodiments, the PI3K inhibitor also inhibits mTOR.

In particular embodiments, the PI3K inhibitor is SF1126, SF1101, BEZ235, BKM120, BYL719, BGT-226, XL-147, GDC-0941, ZSTK-474, PX-866, GDC-0980, PKI-587, PF-04691502, BWT33597, PI-103, CAL-101, GNE-477 or any derivatives thereof.

In some embodiments, the composition is formulated as a pill, a tablet or a capsule.

In other embodiments, the composition is formulated as a syrup, emulsion, or suspension.

In particular embodiments, the composition is formulated as a solid dispersion.

In one embodiment, the solid dispersion is a spray dried dispersion.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The methods described herein for treating a disorder comprises administering to a subject, such as a human androgen receptor antagonist and/or a 17α-hydroxylase/C₁₇₋₂₀-lyase inhibitor (CYP17 inhibitor) as described herein in addition to at least one other therapeutic agent. In some embodiments, the disorder is a neoplastic disorder. In some embodiments, the neoplastic disorder is a cancer. In some embodiments, the CYP17 inhibitor is a 17-heteroarylsteroid compound. In some embodiments, the 17-heteroarylsteroid compound is Compound 1, also referred to as galaterone. In some embodiments, the other therapeutic agent is an anti-cancer agent, a pro-apoptotic agent, or an inhibitor of the PI3K/Akt/mTOR signaling pathway. In some embodiments, the additional therapeutic agent is a direct or indirect inhibitor of an enzyme. In some embodiments, the compositions described herein comprise a 17-heteroarylsteroid compound and at least one additional therapeutic agent. In some embodiments, other anti-cancer treatments, such as administration of one or more other anti-cancer agents, radiotherapy, chemotherapy, photodynamic therapy, surgery, or other immunotherapy, are used with the methods and compositions of the invention.

### DEFINITIONS

As used herein, and unless otherwise defined, the following terms have the meanings provided:

“Neoplastic disorder” refers to a disorder characterized by the abnormal proliferation of cells in the body. In a neoplastic disorder, the growth of neoplastic cells often exceeds, and is not coordinated with, that of the normal tissues around it. In some cases, the growth causes a lump or tumor. Neoplasms may be benign, pre-malignant (carcinoma in situ) or malignant (cancer). In some embodiments, the neoplasm is endometriosis.

“Cancer” refers to the growth, division or proliferation of abnormal cells in the body. Cancers that can be treated with the methods and the compositions described herein include, but are not limited to, prostate cancer, breast cancer, adrenal cancer, leukemia, lymphoma, myeloma, Waldenstrom's macroglobulinemia, monoclonal gammopathy, benign monoclonal gammopathy, heavy chain disease, bone and connective tissue sarcoma, brain tumors, thyroid cancer, pancreatic cancer, pituitary cancer, eye cancer, endometrial cancer, vaginal cancer, vulvar cancer, cervical cancer, uterine cancer, ovarian cancer, esophageal cancer, stomach cancer, colon cancer, rectal cancer, liver cancer, gallbladder cancer, cholangiocarcinoma, lung cancer, testicular cancer, penile cancer, oral cancer, skin cancer, kidney cancer, Wilms' tumor and bladder cancer.

“Recurring cancer” means cancer that has returned after a patient has been earlier diagnosed with cancer, has undergone treatment and/or had been previously diagnosed as cancer-free.

“Relapse cancer” means cancer that was at one time responsive to an anti-cancer treatment, but has become no longer responsive to such treatment or is no longer responding sufficiently to such treatment.

“Refractory cancer” means a cancer that is not responding to an anti-cancer treatment or cancer that is not responding sufficiently to an anti-cancer treatment, including recurring or relapse cancer.

“Treat,” “treating” and “treatment” include the eradication, removal, modification, management or control of a tumor or primary, regional, or metastatic cancer cells or tissue and the minimization or delay of the spread of cancer. In some embodiments, treatment can reduce tumor growth or spread, including preventing any increase in tumor volume. In other embodiments, treatment can decrease tumor volume. In some embodiments, treatment can increase the life span or life quality of a subject with cancer. In some embodiments, the criteria used to determine tumor response to treatment follows Response Evaluation Criteria in Solid Tumors (RECIST) guidelines.

“Subject” means an animal, including but not limited to a mammal, such as a human, monkey, cow, horse, sheep, pig, chicken, turkey, quail, cat, dog, mouse, rat, rabbit, or guinea pig. In one embodiment the subject is a mammal and in another embodiment the subject is a human. In some embodiments, the subject is an adult male or an adult female. In some embodiments, the subject is a male of age about 30 years to about 85 years. In some embodiments, the subject is a female of age about 30 years to about 85 years. In some embodiments, the subject has or is susceptible to having cancer. In some embodiments, the subject has or is susceptible to having a tumor. In some embodiments, the subject is castrated. In some embodiments, the subject is non-castrated. A test subject refers to a subject used for testing the composition or methods of the instant invention, such as for clinical, in vivo, or in vitro studies. In some embodiments, “test subject” can refer to models for cancer study, including but not limited to animal models susceptible to having a tumor, xenografts of cancerous cells into a subject, and in vitro cell cultures.

The term “inhibitor” refers to a compound or therapeutic agent that is able to directly or indirectly inhibit a biological function of a target protein or complex. For example, an inhibitor can act by inhibiting activation of a target protein, by reducing expression of a target protein, or preferably by directly binding to a target protein.

A PI3K inhibitor is an inhibitor of at least one member of the phosphatidylinositol-3-kinase (PI3K) family. Phosphatidylinositide 3-kinases (PI 3-kinases or PI3Ks) are a family of enzymes involved in cellular functions such as cell growth, proliferation, differentiation, motility, survival and intracellular trafficking, which in turn are involved in cancer. The PI3K family includes PIK3C2A, PIK3C2B, PIK3C2G, PIK3C3, PIK3CA, PIK3CB, PIK3CG, PIK3CD, PIK3R1, PIK3R2, PI3KR3, PIK3R4, PIK3R5, and PIK3R6.

An mTOR inhibitor is an inhibitor of at least one mammalian target of rapamycin (mTOR) complex. mTOR exists in at least 2 distinct multiprotein complexes, mTORC1 (described as raptor-mTOR complex) and mTORC2 (described as rictor-mTOR complex). The mTORC1 complex is composed of mTOR, GβL and raptor proteins and binds to FKBP12-rapamycin. mTORC1 is a rapamycin-sensitive complex as its kinase activity is inhibited by FKB 12-rapamycin in vitro. The mTORC2 complex is composed of mTOR, GβL and rictor proteins and it does not bind to FKBP12-rapamycin complex. mTORC2 is a rapamycin-insensitive complex as its kinase activity is not inhibited by FKBP12-rapamycin complex in vitro.

A “selective inhibitor” is an inhibitor that selectively inhibits a target protein compared to off-target proteins. The potency of an inhibitor can be measured, for example, by determining the half maximal inhibitory concentration to inhibit a target protein, complex, or function (IC50). In some embodiments, IC50 can be determined by in vitro assays, such as by measuring inhibition using different concentrations of an inhibitor and determining a dose-response curve. In some embodiments, an inhibitor is a selective inhibitor if it inhibits its selective target with an IC50 that is about 2, 3, 4, 5, 6, 7, 8, 9, 10, 15, 20, 25, 30, 35, 40, 45, 50, 100, or 1000 times less than its IC50 value against a non-selective target.

“17α-Hydroxylase/C17,20-lyase inhibitor” refers to an inhibitor of 17α-hydroxylase/C17,20-lyase, an analog thereof, derivative thereof, metabolite thereof or pharmaceutically-acceptable salt thereof. Also, unless otherwise noted, reference to a particular CYP17 inhibitor can include analogs, derivatives, metabolites or pharmaceutically-acceptable salts of such particular 17α-hydroxylase/C17,20-lyase inhibitor.

“Hormonal agent” includes, but is not limited to, “androgen ablation agents” and “estrogen ablation agents,” and, whether used as “a hormonal ablation agent” or “the hormonal ablation agent,” “hormonal ablation agent” should not be interpreted as being limited to the inclusion of a single hormonal ablation agent.

“Anti-cancer agent” refers to any therapeutic agent that directly or indirectly kills cancer cells or directly or indirectly prohibits, stops or reduces the proliferation of cancer cells. It should be noted that, even though the phrase “anti-cancer agent” may be written as a singular noun, for example, “an anti-cancer agent” or “the anti-cancer agent,” the phrase “anti-cancer agent” should be interpreted as referring to one or more anti-cancer agents.

The terms “effective amount” and “therapeutically effective amount” refer to an amount of a therapeutic agent that is sufficient to affect the intended therapeutic effect for a disease condition, including but not limited to cancer treatment. The terms also apply to a dose that induces a desired response in target cells, both in a subject and in vitro. Such responses include but are not limited to effects on cell survival, apoptosis, signaling activity, protein expression or activity, and proliferation. The effective amount can vary depending on the application, such as the subject (including the species, weight, age, and other factors), the means or method of administration, the composition, the target condition (including, for example, type and severity of the condition), the compound(s) used, and other variables. A “sub-therapeutic” amount of a compound or therapeutic agent as described herein is an amount less than the effective amount of that agent. However, when combined with another agent, compound, or therapy, a sub-therapeutic amount can provide an desired response in a target, such as through synergistic effects.

A “synergistic effect” refers to any effect observed by treating a subject or test subject with at least two therapeutic agents in combination, such as a 17-heteroarylsteroid and an additional therapeutic agent that is greater than the additive effects observed when treating a subject or test subject with each agent alone.

“Pharmaceutically-acceptable salt”, refers to any pharmaceutical salt suitable for administration to a subject. Non-limiting examples of pharmaceutically-acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, formates, isobutyrates, caproates, heptanoates, propiolates, oxalates, malonates, succinates, suberates, sebacates, fumarates, maleates, butyne-1,4-dioates, hexyne-1,6-dioates, malates, benzoates, chlorobenzoates, methylbenzoates, dinitrobenzoates, hydroxybenzoates, methoxybenzoates, phthalates, sulfonates, xylenesulfonates, phylacetates, phenylpropionates, phenylbutyrates, citrates, lactates, gamma-hydroxybutyrates, glycollates, tartrates, alkanesulfonates (e.g., methane-sulfonate or mesylate), trifluoromethylsulfonates, propanesulfonates, naphthalene-1-sulfonates, naphthalene-2-sulfonates, and mandelates. Several of the pharmaceutically-approved salts are listed in Remington: The Science and Practice of Pharmacy, Mack Publishing Co., Easton, Pa.

The following terms have the following meanings, unless otherwise specified.

Alkyl is a C1-C12-straight, C1-C12-branched, or C1-C12-cyclic carbogenic group, which is optionally substituted independently at each position. Non-limiting examples of substituents include hydroxyl, methoxy, ethoxy, sulfhydryl, methyl mercapto, ethylmercapto, fluorine, chlorine, bromine, iodine, aryl, and heteroaryl.

Aryl is a mono- or polycyclic aromatic ring system, which is optionally substituted independently at each position. Non-limiting examples of aryl include phenyl, naphthyl, indenyl, fluorenyl, phenanthrenyl, azulenyl, or C5-C10 aromatic groups. Non-limiting examples of substituents include hydroxyl, methoxy, ethoxy, sulfhydryl, methylmercapto, ethylmercapto, fluorine, chlorine, bromine, iodine, oxo, or heteroaryl.

Heteroaryl is a mono- or polycyclic aromatic system, which is optionally substituted independently at each position, containing at least one ring heteroatom selected from nitrogen, oxygen, and sulfur. Non-limiting examples of substituents include hydroxyl, methoxy, ethoxy, sulfhydryl, methyl mercapto, ethylmercapto, fluorine, chlorine, bromine, iodine, oxo and aryl. Non-limiting examples of heteroaryl include furan, thiophene, pyrrole, pyrrazole, imidazole, oxazole, isoxazole, thiazole, isothiazole, triazole, thiadiazole, oxadiazole, pyridine, pyrimidine, pyrazine, pyridazine, triazine, indole, carbazole, benzofuran, benzothiphene, benzothiazole, benzimidazole, pyrrolopyrimidine, pyrazolopyrimidine, indazole, quinoline, isoquinoline, cinnoline, phthalazine, and heteroaryl groups containing from five-to-twelve or ring atoms.

Aralkyl refers to an aryl group that is distally attached via an alkyl group, for example, benzyl.

Alkylaryl refers to an alkyl group that is distally attached via an aryl group, for example, o-, m-, or p-toluoyl.

Polyalkoxyl is poly(propylene glycol) or poly(ethylene glycol), wherein the monomers are repeated 2-100 times, where such polyalkoxy groups may be defined by the precise range of repeating units (e.g., 35-40), by the targeted peak of envelope distribution in the repeating units (e.g., 114 from PEG5000), or by a selection for solubility or physical properties. Polyalkoxyl groups may also be “capped” by an alkyl group (MPEG5000 for methoxy-PEG5000) or an aryl group, such as phenyl (polyalkoxylaryl).

Methods of Administration and Treatment Methods

A CYP17 inhibitor and an additional therapeutic agent as described herein can be used in the preparation of medicaments for the treatment of a disorder, including but not limited to cancer or cancer-related conditions. In some embodiments, the compound can be used for the treatment of diseases or conditions in which steroid hormone nuclear receptor activity contributes to the pathology and/or symptoms of the disease. In some embodiments, the compound can be used for the treatment of breast cancer or prostate cancer, such as castration-resistant prostate cancer. In addition, a method for treating any of the diseases or conditions described herein in a subject in need of such treatment, involves administration of pharmaceutical compositions containing at least one compound of the invention, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

The compositions containing the compounds and/or additional therapeutic agents described herein can be administered for prophylactic and/or therapeutic treatments. In therapeutic applications, the compositions are administered to a patient already suffering from a disease or condition, in an amount sufficient to cure or at least partially arrest the symptoms of the disease or condition, or to cure, heal, improve, or ameliorate the condition itself. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

Compositions containing the agents described herein can be used to treat, for example, a steroid hormone disorder, disease or condition selected from: primary and secondary hyperaldosteronism, increased sodium retention, increased magnesium and potassium excretion (diuresis), increased water retention, hypertension (isolated systolic and combined systolic/diastolic), inflammation, malignancies such as leukemias and lymphomas, Cushing's syndrome, congenital adrenal hyperplasia, polycystic ovarian syndrome, endometrial cancer, endometriosis, cervical cancer, hypocalcaemia, hyperglycemia, endometriosis, chronic primary adrenal insufficiency, secondary adrenal insufficiency, alopecia, prostate cancer, benign prostatic hyperplasia, alopecia, anorexia nervosa, breast cancer, AIDS, cachexia, for hormone replacement therapy (HRT), employed in male contraception, for male reproductive conditions, primary or secondary male hypogonadism, testicular cancer, ovarian cancer, lung cancer, cardiovascular diseases, osteoporosis, bone loss, abnormally increased bone turnover, metastatic bone disease, hypercalcemia of malignancy, gynecomastia, hirsutism, oligomenorrhea, amenorrhea, anovulation, androgenic alopecia, hypergonadism, excessive acne, or virilization in a patient in need of such treatment, the method comprising administering to the patient an effective amount of a compound described herein, or a tautomer, prodrug, solvate, or salt thereof.

In some embodiments, the methods described herein can be used to treat a neoplastic disorder. In some embodiments, the neoplastic disorder is a cancer. In preferred embodiments the cancer comprises a solid tumor. In some embodiments, the tumor is heterogeneous. Heterogeneous tumors can comprise multiple populations of cells that can, for example, be distinguished by differences in morphology, genetic sequence, epigenetic regulation, or other characteristics known in the art. In some embodiments, the methods described herein can be used to treat a steroid hormone-mediated cancer. In certain embodiments, the steroid hormone mediated cancer is prostate cancer. In other embodiments, the methods described herein can be used to treat castration resistant prostate cancer. In certain embodiments, the steroid hormone mediated cancer is breast cancer. In certain embodiments, the steroid hormone mediated cancer is endometrial cancer. In certain embodiments, steroid hormone mediated cancer is ovarian cancer. In certain embodiments, the steroid hormone mediated cancer is testicular cancer. In certain embodiments, the steroid hormone mediated cancer is cervical cancer.

In some embodiments, the invention described herein provides a method for treating a disease condition, such as a cancer condition. The method generally comprises administering to a subject having such a disease condition a combination of multiple agents, an example of which is a combination comprising a CYP17 inhibitor and an additional therapeutic agent. In some embodiments, the CYP17 inhibitor is a 17-heteroarylsteroid. In some embodiments, the 17-heteroarylsteroid compound is Compound 1. In some embodiments, the additional therapeutic agent is an inhibitor of the PI3K/Akt/mTOR pathway. For example, the additional therapeutic agent can be a pan-PI3K inhibitor; a class I-specific PI3K inhibitor; an inhibitor of one or more specific PI3K enzymes, such as PI3Kα, PI3Kβ, PI3Kγ, PI3Kδ, or some combination thereof; an mTOR inhibitor; or a specific inhibitor of mTORC1 and/or mTORC2.

Dosages of the agents can vary depending on the type of additional therapeutic agent employed, on the specific CYP17 inhibitor employed, on the disease or condition being treated and so forth. In some cases, sub-therapeutic amounts of one or both compounds can be used. In other cases, therapeutically effective amounts of one or both compounds can be used. In addition, the compounds described herein may be administered either simultaneously or sequentially. If administered sequentially, the attending physician can decide on the appropriate sequence of administering the compound and the additional therapeutic agent. In some embodiments, the CYP17 inhibitor compound and the additional therapeutic agent can be administered on alternating days, or with alternating meals. Preferably, the compound and the therapeutic agent are both present in the subject being treated, regardless of the timing or method of administration.

In any case, the agents may be administered in any order or even simultaneously. If simultaneously, the agents may be provided in a single, unified form, or in multiple forms (by way of example only, as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents. Multiple therapeutic combinations are envisioned.

The combination treatment of the invention can be administered before, during or after the occurrence of a disease or condition, and the timing of administering treatment can vary. Thus, for example, the compounds can be used as a prophylactic and can be administered continuously to subjects with a propensity to conditions or diseases in order to prevent the occurrence of the disease or condition. The compounds and compositions can be administered to a subject during or as soon as possible after the onset of the symptoms or after diagnosis. The initial administration can be via any route practical, such as, for example, an intravenous injection, a bolus injection, infusion over 5 minutes to about 5 hours, a pill, a capsule, transdermal patch, buccal delivery, and the like, or a combination thereof. A compound is preferably administered as soon as is practicable after the onset of a disease or condition is detected or suspected, and for a length of time necessary for the treatment of the disease, such as, for example, from about 1 month to about 3 months. The length of treatment can vary for each subject, and the length can be determined using the known criteria. For example, the compound or a formulation containing the compound can be administered for at least 2 weeks, preferably about 1 month to about 3 years, and in some embodiments from about 1 month to about 10 years.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The daily dosages appropriate for Compound 1 or any other CYP17 inhibitor described herein can be, for example, from about 0.03 to about 175 mg/kg body weight, preferably between about 0.03 to about 60 mg/kg body weight, more preferably between about 25 mg/kg to about 50 mg/kg body weight. In some embodiments, the daily dosage is less than 50 mg/kg body weight. An indicated daily dosage in a larger mammal, including, but not limited to, humans, is in the range from about 1 mg to about 4000 mg, conveniently administered in divided doses, including, but not limited to, up to four times a day or in extended release form. Suitable unit dosage forms for oral administration comprise from about 1 mg to about 4000 mg of the 17α-hydroxylase/C17,20-lyase inhibitor, preferably from about 325 mg to about 3500 mg of the agent, most preferably from about 900 mg to about 1950 mg of the agent. In some embodiments, a single dose of compounds of the invention is within the range of about 50 mg to about 2,000 mg. In some embodiments, a single dose of compounds of the invention is about 90 mg, about 200 mg, about 250 mg, about 325 mg, about 650 mg, about 975 mg, about 1300 mg, about 1625 mg, or about 1950 mg. In some embodiments, an administration of compounds of the invention of about 90 mg, about 325 mg, about 650 mg, about 975 mg, about 1300 mg, about 1625 mg, or about 1950 mg is given as multiple doses.

The suitable daily dosage of the CYP17 inhibitor depends upon a number of factors, including the nature of the severity of the condition to be treated, the particular compound employed, the route of administration, and the age, weight, and response of the individual subject. In some embodiments, daily dosages of CYP17 inhibitor range from about 0.01 to about 1000 mg/kg/day, from about 0.01 to about 100 mg/kg/day, from about 0.1 mg/kg/day to about 1000 mg/kg/day, or from about 1 mg/kg/day to about 200 mg/kg/day, or from about 10 mg/kg/day to about 200 mg/kg/day, or from about 1 mg/kg/day to about 100 mg/kg/day. In some embodiments, the daily dosage of CYP17 inhibitor is less than 50 mg/kg/day. In some embodiments, the CYP17 inhibitor is administered in a single dose. In some embodiments, the CYP17 inhibitor is administered in multiple doses.

In some embodiments, the CYP17 inhibitor is administered in an amount of greater than about 0.001 mg/day, 0.01 mg/day, 0.1 mg/day, 0.5 mg/day, 1 mg/day, 5 mg/day, 10 mg/day, 25 mg/day, 50 mg/day, 100 mg/day, 250 mg/day, 500 mg/day, or 1000 mg/day. In some embodiments, the CYP17 inhibitor is administered in an amount of less than about 5000 mg/day, 4000 mg/day, 3000 mg/day, 2500 mg/day, 2000 mg/day, 1800 mg/day, 1500 mg/day, or 1000 mg/day. In some embodiments, the CYP17 inhibitor is administered in an amount from about 0.004 mg/day to about 5000 mg/day, or from about 0.04 mg/day to about 3000 mg/day, or from about 0.4 mg/day to about 1500 mg/day. In some embodiments, the CYP17 inhibitor is administered in an amount from about 0.01 mg/day to about 2000 mg/day, or from about 0.1 mg/day to about 2000 mg/day, or from about 1 mg/day to about 2000 mg/day, or from about 10 mg/day to about 2000 mg/day, or from about 20 mg/day to about 2000 mg/day, or from about 50 mg/day to about 2000 mg/day, or from about 100 mg/day to about 1500 mg/day, or from about 5 mg/day to about 1000 mg/day, or from about 5 mg/day to about 900 mg/day, or from about 10 mg/day to about 800 mg/day, or from about 15 mg/day to about 700 mg/day, or from about 20 mg/day to about 600 mg/day, or from about 25 mg/day to about 500 mg/day, or from about 25 mg/day to about 400 mg/day, or from about 25 mg/day to about 300 mg/day, or from about 25 mg/day to about 250 mg/day, or from about 50 mg/day to about 200 mg/day. In some embodiments, the CYP17 inhibitor is administered in a single dose. In some embodiments, the CYP17 inhibitor is administered in multiple doses. In some embodiments, the CYP17 inhibitor is administered over a period of between about 5 minutes and about 30 minutes. In some embodiments, the CYP17 inhibitor is administered less than once a day. In some embodiments, the CYP17 inhibitor is administered one, two, three, four, five, six, seven, eight, nine, ten, or more than ten times per day.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

In cases wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition. In the case wherein the patient's status does improve, upon the doctor's discretion the administration of the compounds may be given continuously or temporarily suspended for a certain length of time. In some cases, upon the doctor's discretion, administration of the CYP17 inhibitor compound can be adjusted without changing administration of the additional therapeutic agent.

Once improvement of the patient's conditions has occurred, a maintenance dose can be administered if necessary. Subsequently, the dosage or the frequency of administration, or both, can be reduced, as a function of the symptoms, to a level at which the improved disease or condition is retained. Patients can, however, require intermittent treatment on a long-term basis upon any recurrence of symptoms.

Additional Therapeutic Agents

In some embodiments, the methods and compositions of the invention comprise from about 0.5 mg to about 10.0 mg of the additional therapeutic agent, e.g., a PI3K or mTOR inhibitor, in a single composition, optionally with one or more excipients, carriers, diluents, etc., is contemplated. For instance, the single unit dosage form may comprise about 250 mg of the CYP17 inhibitor compound and about 0.5 mg, 0.75 mg, 1.0 mg, 1.25 mg, 1.5 mg, 2.0 mg, 2.25 mg, 2.5 mg, 2.75 mg, 3.0 mg, 3.25 mg, 3.5 mg, 3.75 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, or 10.0 mg of the additional therapeutic agent. In some embodiments, about 0.25 to about 10 mg of the additional therapeutic agent is administered per kg of subject's body weight, preferably between about 0.25 and 3.0 mg/kg. In other embodiments, the less than 2.5 mg of the additional therapeutic agent is administered per kg of subject's body weight.

The PI3K/Akt/mTOR inhibitors for use in the methods and compositions of the invention can be specific or non-specific inhibitors of the pathway. In some embodiments, the inhibitor can bind to and inhibit both PI3K and mTOR. In some embodiments, the inhibitor can specifically bind to and inhibit at least one PI3K family member without directly inhibiting mTOR. In some embodiments, the inhibitor can specifically bind to and inhibit at least one member of Class I PI3K family member relative to a Class II or III PI3K family member. In some embodiments, the inhibitor selectively inhibits PI3Kα, PI3Kβ, PI3Kγ, PI3Kδ, or some combination thereof relative to at least one other class I PI3K family member. In some embodiments, the inhibitor can specifically bind to and inhibit mTOR without directly inhibiting a PI3K family member. In some embodiments, the inhibitor specifically inhibits mTORC1 or mTORC2, or both mTORC1 and mTORC2.

In some embodiments, the additional therapeutic agent is an mTOR inhibitor. Examples of mTOR inhibitors include, but are not limited to, rapamycin/sirolimus, everolimus (RAD001; Novartis), temsirolimus (Torisel™: Wyeth), umirolimus, zotarolimus, deforolimus (MK-8669; Merck, Ariad), wortmannin, TOP-216 (Toptarget A/S), TAFA93 (Isotechnika), CCI-779, ABT578, SAR543, ascomycin, FK506 (tacrolimus; Astellas), AP23573, AP23464, AP23841, KU-0063794, INK-128, EX2044, EX3855, EX7518, AZD-8055, AZD-2014, Palomid 529, Pp-242, OSI-027, and any combinations, derivatives or analogues thereof. Rapamycin derivatives are further described in, e.g., U.S. Pat. Nos. 5,258,389; 5,100,883; 5,118,678; 5,151,413; 5,256,790; and 5,120,842; U.S. Patent Publication 2011/0178070; and PCT applications WO 94/09010; WO 92/05179; WO 93/11130; WO 94/02136; WO 94/02485; WO 94/02136; WO 95/16691; WO 96/41807; WO 96/41807; WO 98/02441; WO 01/14387; and WO 95/14023, all herein incorporated by reference.

mTor inhibitors include, but are not limited to, rapamycin and related compounds. Rapamycin is a macrolide produced by Streptomyces which is a potent immunosuppressive agent and has been used clinically to prevent rejection of transplanted organs. Rapamycin and its analogues are also useful as anti-cancer therapeutic agents, and may be used as mTor inhibitors of the invention. The rapamycins useful in embodiments of the invention include compounds that are chemically or biologically modified as derivatives of the rapamycin nucleus, while still retaining immunosuppressive or anti-cancer properties. Accordingly, rapamycins include rapamycin itself, and esters, ethers, carbamates, oximes, hydrazones, and hydroxylamines of rapamycin, as well as rapamycins in which functional groups on the rapamycin nucleus have been modified, for example through reduction or oxidation.

The structure of rapamycin is shown below:

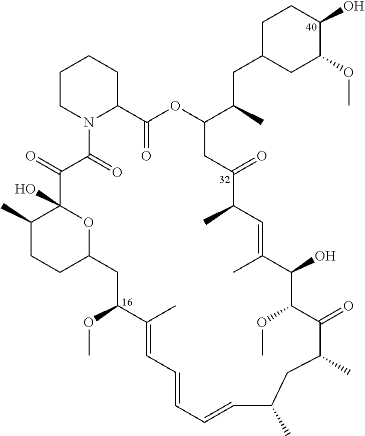 [SMILES: CO[C@H]1CC2CC[C@@H](C)[C@@](O)(O2)C(=O)C(=O)N2CCCCC2C(=O)OC([C@H](C)CC2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

Analogues of the above structure may be prepared, for example, by modifying the structures at positions C-16, C-32 and C-40 as indicated above. For example, the following exemplary substitutions (designated by “R”) are known at position C-40: R=—OP(O)(Me)₂, AP23573 (International Patent Publication Nos. WO 98/02441 and WO 2001/14387); R=—OC(O)C(CH₃)(CH₂OH), temsirolimus (U.S. Pat. No. 5,362,718); R=—OCH₂CH₂OH, everolimus (U.S. Pat. No. 5,665,772); R=—OCH₂CH₂OEt, biolimus; R=-tetrazole, ABT-578 (International Patent Publication No. WO 99/15530). All patents and applications are hereby incorporated by reference. Additional analogues are described as follows: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. Nos. 5,118,678; 5,411,967; 5,434,260; 5,480,988; 5,480,989; 5,489,680); silyl esters (U.S. Pat. No. 5,120,842); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); amidino carbamate esters (U.S. Pat. No. 5,463,048); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462); 32-esters and ethers (U.S. Pat. No. 5,256,790). The preparation of these esters and ethers is disclosed in the patents listed above. All patents and applications are hereby incorporated by reference. Further included are oximes, hydrazones, and hydroxylamines of rapamycin as disclosed in U.S. Pat. Nos. 5,373,014, 5,378,836, 5,023,264, and 5,563,145. The preparation of these oximes, hydrazones, and hydroxylamines is disclosed in the above-listed patents. The preparation of 40-oxorapamycin is disclosed in U.S. Pat. No. 5,023,263. All these patents are hereby incorporated by reference.

In some embodiments, the mTOR inhibitor is not rapamycin or a rapamycin analog. Non-rapamycin analog inhibitors of mTOR include any inhibitors of mTOR that do not comprise the general structure of rapamycin or any rapamycin analogs. Examples of non-rapamycin analog inhibitors of mTOR include other small molecule inhibitors of mTOR, such as, e.g., fused bicyclic compounds (International Patent Publication Nos. WO 2007/61737, WO 2007/87395 and WO 2007/64993), heteroaromatic amines (International Patent Publication No. WO 2001/19828), pyrrolopyrimidine compounds (International Patent Publication No. WO 2005/47289), diphenyl-dihydro-indol-2-one derivatives (International Patent Publication No. WO 2005/97107), and trimethyl-dodeca-triene derivatives (US Patent Publication No. 2007/037887). All of these patents and applications are hereby incorporated by reference.

Other non-rapamycin analog inhibitors of mTOR include the TOR kinase inhibitors (TOR-KIs). TOR-KIs generally target the ATP-binding pocket of the mTOR kinase domain, and can inhibit kinase activity of mTORC1 and/or mTORC2. In some embodiments, the mTOR inhibitor is a TOR-KI. Exemplary TOR-KIs include, e.g., OSI-027, INK-128, BEZ235, LY294002, wortmannin, PI-103, Torin1, PP242, PP30, Ku-0063794, WAY-600, WYE-687, WYE-354, CC-223. Additional TOR-KIs are described in U.S. application Ser. No. 13/192,792, which is hereby incorporated by reference.

In some embodiments, the mTOR inhibitor does not directly bind and inhibit mTOR but indirectly inhibits mTOR through its actions on other constituents of the mTOR pathway (e.g., an mTOR pathway inhibitor). By way of example only, AMPK is a signaling molecule that inhibits mTORC1 indirectly by activation of TSC1/2 and directly by inhibiting the Raptor component of mTORC1. Thus, agents that activate AMPK, such as, e.g., metformin, can be used in the present invention as an mTOR pathway inhibitor. By way of other example, the signaling protein AKT causes disinhibition of mTORC1, therefore, AKT inhibitors can be used in the present invention as an mTOR pathway inhibitor. Exemplary AKT inhibitors include, e.g., AZD5363, GDC-0068, MK2206, Perifosine, RX-0201, PBI-05204, GSK2141795, SR13668. Additional AKT inhibitors are described in US Application Pub. Nos. 20100009397, 20070185152, and U.S. Pat. Nos. 6,960,584, 7,098,208, 7,223,738, 7,304,063, 7,378,403, 7,396,832, 7,399,764, 7,414,055, 7,544,677, 7,576,209, 7,579,355, 7,589,068, 7,638,530, 7,655,649, 7,705,014, 7,750,151, 7,943,732, 8,003,643, 8,003,651, 8,008,317, 8,168,652, 8,263,357, 8,273,782, 8,324,221, all of which are hereby incorporated by reference.

In some embodiments, the mTOR inhibitor is capable of inhibiting both mTORC1 and mTORC2 (mTORC1/mTORC2 inhibitors). In some embodiments, the mTORC1/mTORC2 inhibitor binds to and inhibits both mTORC1 and mTORC2 with an IC50 value of about or less than a predetermined value, as ascertained by an in vitro assay. In some embodiments, the mTOR inhibitor inhibits both mTORC1 and mTORC2 with an IC50 value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.2 μM or less, 1.3 μM or less, 1.4 μM or less, 1.5 μM or less, 1.6 μM or less, 1.7 μM or less, 1.8 μM or less, 1.9 μM or less, 2 μM or less, 5 μM or less, 10 μM or less, 15 μM or less, 20 μM or less, 25 μM or less, 30 μM or less, 40 μM or less, 50 μM or less, 60 μM or less, 70 μM or less, 80 μM or less, 90 μM or less, 100 μM or less, 200 μM or less, 300 μM or less, 400 μM or less, or 500 μM or less.

In some embodiments, the mTORC1/mTORC2 inhibitor is a TOR-KI.

Exemplary TOR-KIs are described above. In other embodiments, the mTORC1/mTORC2 inhibitor is not a TOR-KI. By way of example only, DEPTOR is an endogenous protein that binds to both mTORC1 and mTORC2 to inhibit their activity, thus, agents that increase DEPTOR expression and/or signaling can be used in the present invention as an mTORC1/mTORC2 inhibitor.

In other embodiments, the mTOR inhibitor selectively inhibits mTORC1 as compared to mTORC2. In some embodiments, the mTORC1 inhibitor binds to and inhibits mTORC1 with an IC50 value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.2 μM or less, 1.3 μM or less, 1.4 μM or less, 1.5 μM or less, 1.6 μM or less, 1.7 μM or less, 1.8 μM or less, 1.9 μM or less, 2 μM or less, 5 μM or less, 10 μM or less, 15 μM or less, 20 μM or less, 25 μM or less, 30 μM or less, 40 μM or less, 50 μM or less, 60 μM or less, 70 μM or less, 80 μM or less, 90 μM or less, 100 μM or less, 200 μM or less, 300 μM or less, 400 μM or less, or 500 μM or less, wherein said IC50 value is at least 2, 3, 4, 5, 6, 7, 8, 9, 10, 15, 20, 25, 30, 35, 40, 45, 50, 100, or 1000 times less than its IC50 value against mTORC2, as ascertained by an in vitro assay.

In some embodiments, the mTOR inhibitor also inhibits PI3K (mTOR/PI3K inhibitor). In some embodiments, the mTOR/PI3K inhibitor is a TOR-KI/PI3K inhibitor. In some embodiments, the TOR-KI/PI3K inhibitor inhibits at least one mTOR complex and at least one PI3K family member with an IC50 of 1 tiM, 2 tiM, 5 tiM, 7 tiM, 10 tiM, 20 tiM, 30 tiM, 40 tiM, 50 tiM, 60 tiM, 70 tiM, 80 tiM, 90 tiM, 100 tiM, 120 nM, 140 tiM, 150 tiM, 160 tiM, 170 tiM, 180 tiM, 190 tiM, 200 tiM, 225 tiM, 250 nM, 275 nM, 300 nM, 325 nM, 350 nM, 375 nM, 400 nM, 425 nM, 450 nM, 475 nM, 500 nM, 550 nM, 600 nM, 650 nM, 700 nM, 750 nM, 800 nM, 850 nM, 900 nM, 950 nM, 1 μM, 1.2 μM, 1.3 μM, 1.4 μM, 1.5 μM, 1.6 μM, 1.7 μM, 1.8 μM, 1.9 μM, 2 μM, 5 μM, 10 μM, 15 μM, 20 μM, 25 μM, 30 μM, 40 μM, 50 μM, 60 μM, 70 μM, 80 μM, 90 μM, 100 μM, 200 μM, 300 μM, 400 μM, or 500 μM or less as ascertained in an in vitro assay, wherein said IC50 value for an mTOR complex is not less than 2, 4, 5, 6, 7, 8, 9, 10, 15, 20, 25, 30, 35, 40, 45, 50, 100, or 1000 times less than its IC50 value against any PI3K family members. In preferred embodiments, said IC50 value for an mTOR complex is not less than 2-10 times less than its IC50 value against any PI3K family members. Exemplary mTOR/PI3K inhibitors, include, e.g., SF1126, BEZ235, XL765, NVP-BGT226, GDC-0941, GDC-0980, GSK2126458, PF-04691502, PF-05212384.

In other embodiments, the mTOR inhibitor selectively inhibits mTORC1 and/or mTORC2 relative to PI3K. In some embodiments, the mTOR inhibitor inhibits both mTORC1 and mTORC2 with an IC50 value of about 1 tiM, 2 tiM, 5 tiM, 7 tiM, 10 tiM, 20 tiM, 30 tiM, 40 tiM, 50 tiM, 60 tiM, 70 tiM, 80 tiM, 90 tiM, 100 tiM, 120 nM, 140 tiM, 150 tiM, 160 tiM, 170 tiM, 180 tiM, 190 tiM, 200 tiM, 225 tiM, 250 nM, 275 nM, 300 nM, 325 nM, 350 nM, 375 nM, 400 nM, 425 nM, 450 nM, 475 nM, 500 nM, 550 nM, 600 nM, 650 nM, 700 nM, 750 nM, 800 nM, 850 nM, 900 nM, 950 nM, 1 μM, 1.2 μM, 1.3 μM, 1.4 μM, 1.5 μM, 1.6 μM, 1.7 μM, 1.8 μM, 1.9 μM, 2 μM, 5 μM, 10 μM, 15 μM, 20 μM, 25 μM, 30 μM, 40 μM, 50 μM, 60 μM, 70 μM, 80 μM, 90 μM, 100 μM, 200 μM, 300 μM, 400 μM, or 500 μM or less as ascertained by an in vitro assay, and said IC50 value is at least 2, 3, 4, 5, 6, 7, 8, 9, 10, 15, 20, 25, 30, 35, 40, 45, 50, 100, or 1000 times less than its IC50 value against any PI3K family members. Exemplary compounds and compositions of selective mTORC1/mTORC2 inhibitors over PI3K are described in U.S. patent application Ser. No. 13/003,562, which is hereby incorporated by reference.

In some embodiments, the mTor inhibitor for use according to the invention is BEZ235. The structure of BEZ-235 is shown below:

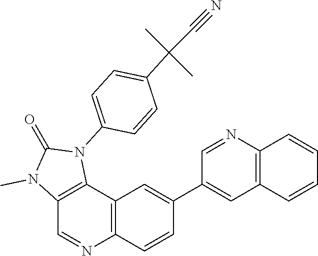 [SMILES: Cn1c(=O)n(-c2ccc(C(C)(C)C#N)cc2)c2c3cc(-c4cnc5ccccc5c4)ccc3ncc21]

In other embodiments, the mTOR inhibitor is BKM-120. The structure of BKM-120 is shown below:

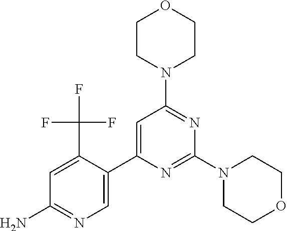 [SMILES: Nc1cc(C(F)(F)F)c(-c2cc(N3CCOCC3)nc(N3CCOCC3)n2)cn1]

In other embodiments, the mTOR inhibitor is OSI-027. The structure of OSI-027 is shown below:

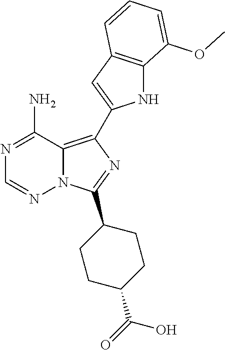 [SMILES: COc1cccc2cc(-c3nc([C@H]4CC[C@H](C(=O)O)CC4)n4ncnc(N)c34)[nH]c12]

In other embodiments, the mTOR inhibitor is XL765. The structure of XL765 is shown below:

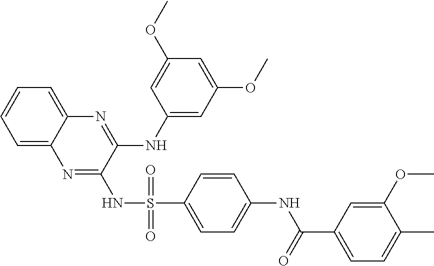 [SMILES: COc1cc(Nc2nc3ccccc3nc2NS(=O)(=O)c2ccc(NC(=O)c3ccc(C)c(OC)c3)cc2)cc(OC)c1]

In some embodiments, the mTOR inhibitor inhibits mTOR through RNA inhibition (RNAi). RNAi refers to cellular processes for the targeted degradation of sequence-specific mRNA. These cellular processes can be manipulated by introducing short double-stranded RNA molecules, such as, e.g., short interfering RNA (siRNA), short hairpin RNA (shRNA), and/or microRNA (miRNA), that are complementary to the mRNA sequence targeted for degradation. Therefore, the mTOR inhibitor of the invention can be an shRNA, siRNA, or miRNA designed to be substantially complementary to components of mTORC1, mTORC2, PI3K, or other components of the mTOR/PI3K pathway. Exemplary RNAi inhibitors of mTOR are described in WO/2008/027855, which is hereby incorporated by reference.

In some embodiments, the additional therapeutic agent is a PI3K inhibitor. In particular embodiments, the PI3K inhibitor is a pan-PI3K inhibitor. In some embodiments, the inhibitor can specifically bind to and inhibit at least one member of Class I PI3K family member relative to a Class II or III PI3K family member. Class I PI3K family members refers to PIK3CA, PIK3CB, PIK3CG, PIK3CD, PIK3R1, PIK3R2, PI3KR3, PIK3R4, PIK3R5, and PIK3R6. Class II and III PI3K family members refer to PIK3C2A, PIK3C2B, PIK3C2G, PIK3C3. In some embodiments, the inhibitor selectively inhibits PIK3CA, PIK3CB, PIK3CG, PIK3CD, or some combination thereof relative to at least one other class I PI3K family member. Examples of PI3K inhibitors include but are not limited to wortmannin, SF1126, SF1101, BEZ235, BKM120, BYL719, BGT-226, XL-147, XL-765, GSK1059615, GDC-0941, ZSTK-474, PX-866, GDC-0980, PKI-587, PF-04691502, BWT33597, PI-103, CAL-101, CAL-120, ONC-21, AEZS-127, ETP-45658, PX-866, LY-294002, GNE-477, and any combinations, derivatives or analogues thereof. Examples of PI3K inhibitors are further described in U.S. Patent Publications 2011/0195966, 2011/0178070, herein incorporated by reference.

In some embodiments, the methods of the invention comprise administering an amount from about 20 mg to about 3500 mg of the CYP17 inhibitor and about 0.25 mg to about 150 mg of the additional therapeutic agent. In some embodiments, the methods of the invention comprise administering an amount from about 20 mg to about 3500 mg of the CYP17 inhibitor and about 0.25 mg to about 250 mg of the additional therapeutic agent.

In some embodiments, the method for the treatment of a cancer in a subject comprises administering about 0.01 mg/kg/day to about 100 mg/kg/day of the CYP17 inhibitor and about 0.1 mg/m² to about 20 mg/m² of the additional therapeutic agent. In some embodiments, the additional therapeutic agent is administered over a period of between about 5 to about 30 minutes. In some embodiments, the additional therapeutic agent is administered less than once a day. In some embodiments, the additional therapeutic agent is administered one, two, three, four, five, six, seven, eight, nine, ten, or more than ten times per day.

In some embodiments, a method for the treatment of cancer in a subject includes administering 0.01 mg/kg/day to about 100 mg/kg/day of the 17α-hydroxylase/C17,20-lyase inhibitor and about 0.01 to about 100 mg/kg/day of the additional therapeutic agent.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

In cases wherein the patient's condition does not improve, upon the doctor's discretion the administration of the additional therapeutic agent may be administered chronically, that is, for an extended period of time, including throughout the duration of the patient's life in order to ameliorate or otherwise control or limit the symptoms of the patient's disease or condition. In the case wherein the patient's status does improve, upon the doctor's discretion the administration of the additional therapeutic agent may be given continuously or temporarily suspended for a certain length of time. In some cases, upon the doctor's discretion, administration of the additional therapeutic agent can be adjusted without changing administration of the CYP17 inhibitor compound.

Once improvement of the patient's conditions has occurred, a maintenance dose can be administered if necessary. Subsequently, the dosage or the frequency of administration, or both, can be reduced, as a function of the symptoms, to a level at which the improved disease or condition is retained. Patients can, however, require intermittent treatment on a long-term basis upon any recurrence of symptoms.

In addition, the combination treatment of the invention may also be used in combination with procedures that may provide additional or synergistic benefit to the patient. By way of example only, patients are expected to find therapeutic and/or prophylactic benefit in the methods described herein, wherein pharmaceutical composition of the invention and/or combinations with other therapeutics are combined with genetic testing to determine whether that individual is a carrier of a mutant gene that is known to be correlated with certain diseases or conditions.

In certain instances, it may be appropriate to administer the agents described herein (or a pharmaceutically acceptable salts, pharmaceutically acceptable N-oxides, pharmaceutically active metabolites, pharmaceutically acceptable prodrugs, and pharmaceutically acceptable solvates thereof) in combination with a third therapeutic agent. By way of example only, if one of the side effects experienced by a patient upon receiving the agents of the invention herein is inflammation, then it may be appropriate to administer an anti-inflammatory agent in combination with the compound and additional therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering the combination treatment described herein with another therapeutic agent (which also includes a therapeutic regimen) that also has therapeutic benefit. In any case, regardless of the disease or condition being treated, the overall benefit experienced by the patient may simply be additive or synergistic.

Suitable compounds that may be used in addition to combination treatment described herein include, but are not limited to, hormone ablation agents, anti-androgen agents/anti-androgens, anti-estrogen agents/anti-estrogens, differentiating agents, anti-neoplastic agents, kinase inhibitors, anti-metabolite agents, alkylating agents, antibiotic agents, immunological agents, interferon-type agents, intercalating agents, growth factor inhibitors, including but not limited to epidermal growth factor inhibitors, cell-cycle inhibitors, enzymes, topoisomerase inhibitors, biological response modifiers, mitotic inhibitors, matrix metalloprotease inhibitors, and genetic therapeutics. The amount of the additional agents can be an amount that is sufficient to treat the cancer or other disease, whether administered alone or in combination with the combination treatment of the invention described herein. Examples of some of the above classes of agents are listed below for purposes of illustration and not for purposes of limitation, as these examples are not all-inclusive. Many of the examples below could be listed in multiple classes of agents for treating steroid hormone mediated disorders and are not restricted in any way to the class in which they are listed.

In some embodiments, the combination treatment of the invention may be administered with an hormonal ablation agent, such as deslorelin, leuprolide, goserelin or triptorelin. In some embodiments, the amount of the hormonal ablation agent administered can be an amount that is sufficient to treat the disease condition, whether administered alone or in combination with Compound I and the PI3K/Akt/mTOR inhibitor described herein.

Suitable anti-androgen agents include but are not limited to bicalutamide, flutamide and nilutamide. In some embodiments, the amount of the anti-androgen agent administered can be an amount that is sufficient to treat the disease condition, whether administered alone or in combination with the CYP17 inhibitor compound and the PI3K/Akt/mTOR inhibitor described herein.

Suitable anti-estrogen agents include but are not limited to tamoxifen, raloxifene, 4-hydroxytamoxifen (afimoxifene), clomifene, arzoxifene, bazedoxifene, ormeloxifene, and toremifene. In some embodiments, the amount of the anti-estrogen agent administered can be an amount that is sufficient to treat the disease condition, whether administered alone or in combination with the CYP17 inhibitor compound and the PI3K/Akt/mTOR inhibitor described herein.

In another embodiment, the CYP17 inhibitor compound and the additional therapeutic agent may be administered with a differentiating agent. Suitable differentiating agents include, but are not limited to, polyamine inhibitors; vitamin D and its analogs, such as calcitriol, doxercalciferol and seocalcitol; metabolites of vitamin A, such as ATRA; retinoic acid; retinoids; short-chain fatty acids; phenylbutyrate; and nonsteroidal anti-inflammatory agents. In some embodiments, the amount of the differentiating agent administered can be an amount that is sufficient to treat the disease condition, whether administered alone or in combination with the CYP17 inhibitor compound and the PI3K/Akt/mTOR inhibitor described herein.

In another embodiment, the CYP17 inhibitor compound and the additional therapeutic agent may be administered with an anti-neoplastic agent, including, but not limited to, tubulin interacting agents, topoisomerase inhibitors and agents, acitretin, alstonine, amonafide, amphethinile, amsacrine, ankinomycin, anti-neoplaston, aphidicolin glycinate, asparaginase, baccharin, batracylin, benfluoron, benzotript, bromofosfamide, caracemide, carmethizole hydrochloride, chlorsulfaquinoxalone, clanfenur, claviridenone, crisnatol, curaderm, cytarabine, cytocytin, dacarbazine, datelliptinium, dihaematoporphyrin ether, dihydrolenperone, dinaline, distamycin, docetaxel, elliprabin, elliptinium acetate, epothilones, ergotamine, etoposide, etretinate, fenretinide, gallium nitrate, genkwadaphnin, hexadecylphosphocholine, homoharringtonine, hydroxyurea, ilmofosine, isoglutamine, isotretinoin, leukoregulin, lonidamine, merbarone, merocyanlne derivatives, methylanilinoacridine, minactivin, mitonafide, mitoquidone, mitoxantrone, mopidamol, motretinide, N-(retinoyl)amino acids, N-acylated-dehydroalanines, nafazatrom, nocodazole derivative, ocreotide, oquizanocine, paclitaxel, pancratistatin, pazelliptine, piroxantrone, polyhaematoporphyrin, polypreic acid, probimane, procarbazine, proglumide, razoxane, retelliptine, spatol, spirocyclopropane derivatives, spirogermanium, strypoldinone, superoxide dismutase, teniposide, thaliblastine, tocotrienol, topotecan, ukrain, vinblastine sulfate, vincristine, vindesine, vinestramide, vinorelbine, vintriptol, vinzolidine, and withanolides, cabazitaxel, sipuleucel-T, and enzalutamide. In some embodiments, the amount of the anti-neoplastic agent administered can be an amount that is sufficient to treat the disease condition, whether administered alone or in combination with the CYP17 inhibitor compound and the PI3K/Akt/mTOR inhibitor described herein.

In another embodiment, the CYP17 inhibitor compound and the additional therapeutic agent may be administered with an anti-cancer stem cell therapeutic. Exemplary anti-cancer stem cell therapeutics are described in U.S. Pat. Nos. 8,129,184 and 7,604,947, WIPO Patent Application WO/2011/116344A2, and U.S. patent application Ser. No. 13/055,542, which are hereby incorporated by reference.

The CYP17 inhibitor compound and the additional therapeutic agent may also be used with a kinase inhibitor, including p38 inhibitors and CDK inhibitors, TNF inhibitors, metallomatrix proteases (MMP) inhibitors; COX-2 inhibitors, including celecoxib, rofecoxib, parecoxib, valdecoxib, and etoricoxib; SOD mimics; or αᵥ,β₃-inhibitors. In some embodiments, the amount of the kinase inhibitor administered can be an amount that is sufficient to treat the disease condition, whether administered alone or in combination with the CYP17 inhibitor compound and the PI3K/Akt/mTOR inhibitor described herein.

In another embodiment, the CYP17 inhibitor compound and the additional therapeutic agent may be administered with an anti-metabolite agent. Suitable anti-metabolite agents may be selected from, but are not limited to, 5-FU-fibrinogen, acanthifolic acid, aminothiadiazole, brequinar sodium, carmofur, cyclopentyl cytosine, cytarabine phosphate stearate, cytarabine conjugates, dezaguanine, dideoxycytidine, dideoxyguanosine, didox, doxifluridine, fazarabine, floxuridine, fludarabine phosphate, 5-fluorouracil, N-(2′-furanidyl)-5-fluorouracil, isopropyl pyrrolizine, methobenzaprim, methotrexate, norspermidine, pentostatin, piritrexim, plicamycin, thioguanine, tiazofurin, trimetrexate, tyrosine kinase inhibitors, and uricytin. In some embodiments, the amount of the anti-metabolite agent administered can be an amount that is sufficient to treat the disease condition, whether administered alone or in combination with the CYP17 inhibitor compound and the PI3K/Akt/mTOR inhibitor described herein.

In still another embodiment, the CYP17 inhibitor compound and the additional therapeutic agent may be administered with an alkylating agent, that may be selected from, but not limited to, aldo-phosphamide analogues, altretamine, anaxirone, bestrabucil, budotitane, carboplatin, carmustine, chlorambucil, cisplatin, cyclophosphamide, cyplatate, diphenylspiromustine, diplatinum cytostatic, elmustine, estramustine phosphate sodium, fotemustine, hepsul-fam, ifosfamide, iproplatin, lomustine, mafosfamide, mitolactol, oxaliplatin, prednimustine, ranimustine, semustine, spiromustine, tauromustine, temozolomide, teroxirone, tetraplatin and trimelamol. In some embodiments, the amount of the alkylating agent administered can be an amount that is sufficient to treat the disease condition, whether administered alone or in combination with the CYP17 inhibitor compound and the PI3K/Akt/mTOR inhibitor described herein.

In yet another preferred embodiment, the CYP17 inhibitor compound and the additional therapeutic agent may be administered with an antibiotic agent. Suitable antibiotic agents may be selected from, but are not limited to, aclarubicin, actinomycin D, actinoplanone, adriamycin, aeroplysinin derivative, amrubicin, anthracycline, azinomycin-A, bisucaberin, bleomycin sulfate, bryostatin-1, calichemycin, chromoximycin, dactinomycin, daunorubicin, ditrisarubicin B, dexamethasone, doxorubicin, doxorubicin-fibrinogen, elsamicin-A, epirubicin, erbstatin, esorubicin, esperamicin-Al, esperamicin-Alb, fostriecin, glidobactin, gregatin-A, grincamycin, herbimycin, corticosteroids, idarubicin, illudins, kazusamycin, kesarirhodins, menogaril, mitomycin, neoenactin, oxalysine, oxaunomycin, peplomycin, pilatin, pirarubicin, porothramycin, prednisone, prednisolone, pyrindanycin A, rapamycin, rhizoxin, rodorubicin, sibanomicin, siwenimycin, sorangicin-A, sparsomycin, talisomycin, terpentecin, thrazine, tricrozarin A, and zorubicin. In some embodiments, the amount of the antibiotic agent administered can be an amount that is sufficient to treat the disease condition, whether administered alone or in combination with the CYP17 inhibitor compound and the PI3K/Akt/mTOR inhibitor described herein.

Alternatively, the CYP17 inhibitor compound and the additional therapeutic agent may also be used with other anti-cancer agents, including but not limited to, acemannan, aclarubicin, aldesleukin, alemtuzumab, alitretinoin, altretamine, amifostine, amsacrine, anagrelide, anastrozole, ancestim, bexarotene, broxuridine, capecitabine, celmoleukin, cetrorelix, cladribine, clotrimazole, daclizumab, dexrazoxane, dilazep, docosanol, doxifluridine, bromocriptine, carmustine, cytarabine, diclofenac, edelfosine, edrecolomab, eflornithine, emitefur, exemestane, exisulind, fadrozole, filgrastim, finasteride, fludarabine phosphate, formestane, fotemustine, gallium nitrate, gemcitabine, glycopine, heptaplatin, ibandronic acid, imiquimod, iobenguane, irinotecan, irsogladine, lanreotide, leflunomide, lenograstim, lentinan sulfate, letrozole, liarozole, lobaplatin, lonidamine, masoprocol, melarsoprol, metoclopramide, mifepristone, miltefosine, mirimostim, mitoguazone, mitolactol, molgramostim, nafarelin, nartograstim, nedaplatin, nilutamide, noscapine, oprelvekin, osaterone, oxaliplatin, pamidronic acid, pegaspargase, pentosan polysulfate sodium, pentostatin, picibanil, pirarubicin, porfimer sodium, raloxifene, raltitrexed, rasburicase, rituximab, romurtide, sargramostim, sizofiran, sobuzoxane, sonermin, suramin, tasonermin, tazarotene, tegafur, temoporfin, temozolomide, teniposide, tetrachlorodecaoxide, thalidomide, thymalfasin, thyrotropin alfa, topotecan, toremifene, trastuzumab, treosulfan, tretinoin, trilostane, trimetrexate, ubenimex, valrubicin, verteporfin, and vinorelbine. In some embodiments, the amount of the additional anti-cancer agent(s) administered can be an amount that is sufficient to treat the disease condition, whether administered alone or in combination with the CYP17 inhibitor compound and the PI3K/Akt/mTOR inhibitor described herein.

The CYP17 inhibitor compound and the additional therapeutic agent may also be administered or combined with steroids, such as corticosteroids or glucocorticoids, non-limiting examples of such suitable steroids including hydrocortisone (cortisol; cyprionate oral; sodium phosphate injection; sodium succinate; cortisone acetate oral or injection forms, etc.), prednisone, prednisolone (e.g., DELTA-CORTEF® prednisolone sodium succinate, prednisolone acetate, prednisolone sodium phosphate, prednisolone tebutate), or dexamethasone (e.g., DECADRON® oral; Decadron®-LA injection, etc.) and combinations thereof. See, e.g., Goodman & Gilman's The Pharmacological Basis of Therapeutics, 10^{th} sup. Edition 2001. In some embodiments, the amount of the steroid administered can be an amount that is sufficient to treat the disease condition, whether administered alone or in combination with the CYP17 inhibitor compound and the PI3K/Akt/mTOR inhibitor described herein.

Pharmacokinetic and Pharmacodynamic Measurements

Pharmacokinetic and pharmacodynamic data can be obtained by known techniques in the art. Due to the inherent variation in pharmacokinetic and pharmacodynamic parameters of drug metabolism in human subjects, appropriate pharmacokinetic and pharmacodynamic profile components describing a particular composition can vary. Typically, pharmacokinetic and pharmacodynamic profiles are based on the determination of the “mean” parameters of a group of subjects. The group of subjects includes any reasonable number of subjects suitable for determining a representative mean, for example, 5 subjects, 10 subjects, 16 subjects, 20 subjects, 25 subjects, 30 subjects, 35 subjects, or more. The “mean” is determined by calculating the average of all subject's measurements for each parameter measured.

The pharmacokinetic parameters can be any parameters suitable for describing the present composition. For example, the Cₘₐₓ can be not less than about 500 ng/ml; not less than about 550 ng/ml; not less than about 600 ng/ml; not less than about 700 ng/ml; not less than about 800 ng/ml; not less than about 880 ng/ml, not less than about 900 ng/ml; not less than about 100 ng/ml; not less than about 1250 ng/ml; not less than about 1500 ng/ml, not less than about 1700 ng/ml, or any other Cₘₐₓ appropriate for describing a pharmacokinetic profile of Compound 1 or any other CYP17 inhibitor compound. In other embodiments, the Cmax is less than 500 ng/ml. In some embodiments wherein the active metabolite is formed in vivo after administration of a drug to a subject; the Cₘₐₓ can be not less than about 500 pg/ml; not less than about 550 pg/ml; not less than about 600 pg/ml; not less than about 700 pg/ml; not less than about 800 pg/ml; not less than about 880 pg/ml, not less than about 900 pg/ml; not less than about 1000 pg/ml; not less than about 1250 pg/ml; not less than about 1500 pg/ml, not less than about 1700 pg/ml, or any other Cₘₐₓ appropriate for describing a pharmacokinetic profile of a compound formed in vivo after administration of the 17α-hydroxylase/C17,20-lyase inhibitor compound to a subject.

The Tₘₐₓ can be, for example, not greater than about 0.5 hours, not greater than about 1.0 hours, not greater than about 1.5 hours, not greater than about 2.0 hours, not greater than about 2.5 hours, or not greater than about 3.0 hours, or any other Tₘₐₓ appropriate for describing a pharmacokinetic profile of the 17α-hydroxylase/C17,20-lyase inhibitor compound.

The AUC_{(0-inf)} can be, for example, not less than about 590 ng·hr/mL, not less than about 1500 ng·hr/mL, not less than about 2000 ng·hr/mL, not less than about 3000 ng·hr/ml, not less than about 3500 ng·hr/mL, not less than about 4000 ng·hr/mL, not less than about 5000 ng·hr/mL, not less than about 6000 ng·hr/mL, not less than about 7000 ng·hr/mL, not less than about 8000 ng·hr/mL, not less than about 9000 ng·hr/mL, or any other AUC_{(0-inf)} appropriate for describing a pharmacokinetic profile of the CYP17 inhibitor compound. In other embodiments, the AUC is less than 590 ng*hr/ml. In some embodiments wherein an active metabolite is formed in vivo after administration of the CYP17 inhibitor compound to a subject; the AUC_{(0-inf)} can be, for example, not less than about 590 pg·hr/mL, not less than about 1500 pg·hr/mL, not less than about 2000 pg·hr/mL, not less than about 3000 pg·hr/mL, not less than about 3500 pg·hr/mL, not less than about 4000 pg·hr/mL, not less than about 5000 pg·hr/mL, not less than about 6000 pg·hr/mL, not less than about 7000 pg·hr/mL, not less than about 8000 pg·hr/mL, not less than about 9000 pg·hr/mL, or any other AUC_{(0-inf)} appropriate for describing a pharmacokinetic profile of a compound formed in vivo after administration of the CYP17 inhibitor compound to a subject.

The plasma concentration of the CYP17 inhibitor compound about one hour after administration can be, for example, not less than about 140 ng/ml, not less than about 425 ng/ml, not less than about 550 ng/ml, not less than about 640 ng/ml, not less than about 720 ng/ml, not less than about 750 ng/ml, not less than about 800 ng/ml, not less than about 900 ng/ml, not less than about 1000 ng/ml, not less than about 1200 ng/ml, or any other plasma concentration of the CYP17 inhibitor compound.

The pharmacodynamic parameters can be any parameters suitable for describing the present composition. For example, the pharmacodynamic profile can exhibit decreases in AR protein or endogenous androgens for, by way of example only, at least about 2 hours, at least about 4 hours, at least about 8 hours, at least about 12 hours or at least about 24 hours. The pharmacodynamic profile can exhibit an inhibition of androgen synthesizing enzymes, including CYP17, for, by way of example only, at least about 2 hours, at least about 4 hours, at least about 8 hours, at least about 12 hours or at least about 24 hours. The pharmacodynamic profile can exhibit reduction of androgen signaling, for, by way of example only, at least about 2 hours, at least about 4 hours, at least about 8 hours, at least about 12 hours or at least about 24 hours.

Toxicity and therapeutic efficacy of such therapeutic regimens can be determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

In another aspect, the invention provides a method of treating prostate cancer in a subject in need thereof, comprising administering to said subject a CYP17 inhibitor and at least one additional therapeutic agent, wherein the additional therapeutic agent is a PI3K inhibitor and/or mTOR inhibitor if a biomarker that indicates the presence or progression of prostate cancer has increased over time. In another embodiment, the method comprises administering CYP17 inhibitor and at least one additional therapeutic agent, wherein the additional therapeutic agent is a PI3K inhibitor and/or mTOR inhibitor, to said subject if a biomarker that indicates the presence or progression of prostate cancer is above a threshold level.

The biomarker can be any analyte that indicates the presence or progression of prostate cancer. The analyte can be, for example, a protein, peptide, amino acid, or nucleic acid molecule such as, e.g., DNA or RNA.

In particular embodiments, the biomarker is a protein that can be detected from a biological sample from said subject. The biological sample can be provided the subject, or provided indirectly through an intermediary, such as, for example, a sample collection service provider or a medical provider. In some embodiments, the biological sample is a bodily fluid sample, e.g., whole blood, plasma, serum, saliva, sweat, tears, sputum, urine, lymphatic fluid, effusions such as, e.g., peritoneal cavity effusion (ascites). In other embodiments, the fluid sample is a rinse of a bodily organ or cavity. Rinses can be obtained from numerous organs, body cavities, passage ways, ducts and glands. Sites that can be rinsed include lungs (bronchial lavage), stomach (gastric lavage), gastrointestinal tract (gastrointestinal lavage), colon (colonic lavage), bladder (bladder irrigation), oral, nasal, sinus cavities, and peritoneal cavity (peritoneal cavity perfusion). In other embodiments, the biological sample is a solid tissue sample. Solid tissue samples may be derived from individuals by any method known in the art, including surgical specimens, biopsies such as, e.g., tumor biopsies, and tissue scrapings.

In particular embodiments, the biomarker is a protein. In particular embodiments, the protein is prostate-specific antigen (PSA). In other embodiments, the biomarker is glutamate, osteopontin, or prostatic acid phosphatase (PAP). Examples of other biomarkers that may be used to indicate the presence or progression of prostate cancer are described in U.S. Pat. No. 7,807,393, US Patent Application Pub. No. 20090221672, WIPO Patent applications WO/2010/028646A1, WO/2012/129408A2, all of which are hereby incorporated by reference.

In some cases, a subject is treated for prostate cancer by administration of a CYP17 inhibitor and a PI3K inhibitor and/or mTOR inhibitor based on an increase in a biomarker that indicates the presence or progression of prostate cancer. Therefore, in some embodiments, the biomarker is detected or measured at a plurality of time points. In some embodiments, the biomarker is detected or measured at about 2, 3, 4, 5, 6, 7, 8, 9, 10, 15, 20, 25, 30, 35, 40, 45, or 50 or more time points. In some embodiments, the biomarker is detected or measured at a first and second time point that are at least 1 day apart, at least 2 days apart, at least 3, days apart, at least 4 days apart, at least 5 days apart, at least 6 days apart, at least a week apart, at least two weeks apart, at least a month apart, or more than a month apart. In a preferred embodiment, the first and second time point is at least one week apart.

In some cases, a subject is treated for prostate cancer by administration of a CYP17 inhibitor and a PI3K inhibitor and/or mTOR inhibitor based on a detected level of a biomarker at or above a set threshold level. In some embodiments, the set threshold level is 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 15 ng/ml, 20 ng/ml, or greater than 20 ng/ml. In a preferred embodiment, the set threshold level is 5 ng/ml.

In some embodiments, the biomarker is detected, qualitatively or quantitatively, by an immunoassay procedure. The immunoassay typically includes contacting a test sample with an agent that specifically binds to or otherwise recognizes the biomarker, and detecting the presence of a complex of the agent bound to the biomarker in the sample. In some embodiments, the agent is an antibody that selectively recognizes and forms a binding complex with the biomarker. The immunoassay procedure may be selected from a wide variety of immunoassay procedures known to the art involving recognition of antibody/antigen complexes, including enzyme immunoassays, which may be competitive or non-competitive, including, e.g., enzyme-linked immunosorbent assays (ELISA), radioimmunoassays (RIA), and Western blots. In some embodiments, more than one biomarker may be detected by multiplex assay. Examples of multiplex assays include, e.g., use of antibody arrays wherein several desired antibodies are placed on a solid support and reacted or otherwise contacted with the test sample. The solid support can be, e.g., a glass bead or plate. Such assays are well known to the skilled artisan and are described, for example, more thoroughly in Antibodies: A Laboratory Manual (1988) by Harlow & Lane Immunoassays: A Practical Approach, Oxford University Press, Gosling, J. P. (ed.) (2001) and/or Current Protocols in Molecular Biology (Ausubel et al.) which is regularly and periodically updated.

In alternative embodiments, the agent that specifically binds to or otherwise recognizes the biomarker is not an antibody. In some embodiments, the agent is any other suitable agent (e.g., a peptide, an aptamer, lectin, or a small organic molecule) that specifically binds a biomarker. In particular embodiments, the agent is an aptamer. Aptamers are nucleic acid-based molecules that bind specific ligands. Methods for making aptamers with a particular binding specificity are known as detailed in U.S. Pat. No. 5,475,096; No. 5,670,637; No. 5,696,249; No. 5,270,163; No. 5,707,796; No. 5,595,877; No. 5,660,985; No. 5,567,588; No. 5,683,867; No. 5,637,459; and No. 6,011,020, which are hereby incorporated by reference.

In another aspect, the invention provides methods for the treatment of a cancer in a subject in need thereof, comprising administering a 17a the invention_{17,20}-lyase inhibitor and an additional therapeutic agent if said subject harbors a mutation or copy number variation in a gene associated with the PI3K/mTOR pathway. In some embodiments, the mutation and/or copy number variation can be a PTEN mutation, a PTEN loss-of-heterozygosity, a PIK3CA mutation, a PIK3CA amplification, an AKT mutation, an AKT amplification, or a P85α mutation. In some embodiments, the mutation is evident in a tumor cell obtained from said subject. In some embodiments, the tumor cell is obtained from a tumor biopsy from said subject. In another embodiment, the mutation is evident in a tissue cell obtained from said subject. In a more particular embodiment, the tissue cell is a prostate cell. In other embodiments, the tumor cell is obtained from circulating tumor cells found in the blood of said subject. In some embodiments, the mutation is evident in nucleic acids originating from a tumor cell. In particular embodiments, the nucleic acids may be cell-free nucleic acids.

The mutation and/or copy number variation can be determined using methods known in the art, such as, by way of example only, cytogenetic techniques such as fluorescent in situ hybridization, comparative genomic hybridization, array comparative genomic hybridization, STR analysis, SNP array, sequencing, such as, e.g., next-generation sequencing.

### Compounds

“17-heteroarylsteroid” compounds include 3-β-hydroxy-17-(1H-benzimidazole-1-yl)androsta-5,16-diene, herein “Compound 1” or “Cpd1”; and abiraterone alcohol, an active pharmaceutical ingredient and plasma enzymatic cleavage product of abiraterone acetate. Compound 1, pharmaceutically acceptable salts, pharmaceutically acceptable N-oxides, pharmaceutically active metabolites, pharmaceutically acceptable prodrugs, pharmaceutically acceptable polymorphs and pharmaceutically acceptable solvates thereof, modulate the activity of steroid hormone nuclear receptors and, as such, are useful, for example, for treating androgen receptor mediated diseases or conditions.

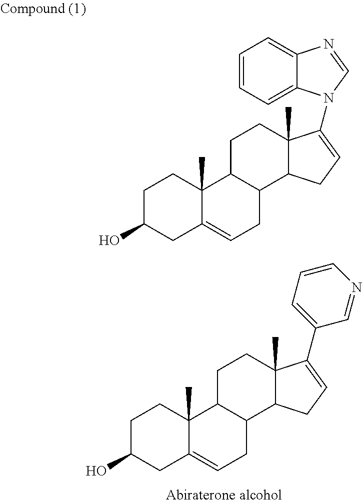 [SMILES: C[C@]12CC[C@H](O)CC1=CCC1C2CC[C@]2(C)C(n3cnc4ccccc43)=CCC12 ; C[C@]12CC[C@H](O)CC1=CCC1C2CC[C@]2(C)C(c3cccnc3)=CCC12]

The invention also contemplates the combination of 17α-hydroxylase/C₁₇₋₂₀-lyase inhibitor compounds with mTOR and/or PI3K inhibitors for the treatment of cancer, wherein the 17α-hydroxylase/C₁₇₋₂₀-lyase inhibitor is not a 17-heteroarylsteroid compound. In some embodiments, the 17α-hydroxylase/C₁₇₋₂₀-lyase inhibitor is TAK-700. The structure of TAK-700 is shown below:

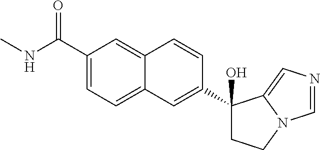 [SMILES: CNC(=O)c1ccc2cc([C@@]3(O)CCn4cncc43)ccc2c1]

### Synthesis of the Compounds

Compound 1 or 3-β-Hydroxy-17-(1H-benzimidazol-1-yl)androsta-5,16-diene) may be synthesized using standard synthetic techniques known to those of skill in the art or using methods known in the art in combination with methods described herein. In additions, solvents, temperatures and other reaction conditions presented herein may vary according to the practice and knowledge of those of skill in the art.

The starting material used for the synthesis of the Compound 1 can be obtained from commercial sources, such as Aldrich Chemical Co. (Milwaukee, Wis.), Sigma Chemical Co. (St. Louis, Mo.), or the starting materials can be synthesized. The compounds described herein, and other related compounds having different substituents can be synthesized using techniques and materials known to those of skill in the art, such as described, for example, in March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference in their entirety). General methods for the preparation of compounds as disclosed herein may be derived from known reactions in the field, and the reactions may be modified by the use of appropriate reagents and conditions, as would be recognized by the skilled person, for the introduction of the various moieties found in the formulae as provided herein.

Compound 1 can be prepared as a pharmaceutically acceptable acid addition salt (which is a type of a pharmaceutically acceptable salt) by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of Compound 1 can be conveniently prepared or formed during the processes described herein. By way of example only, hydrates of Compound 1 can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

Compound 1 includes crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

Compound 1 can be prepared as a prodrug. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they may be easier to administer than the parent drug. Prodrugs may, for instance, be bioavailable by oral administration whereas the parent is not. The prodrug may also have improved solubility in pharmaceutical compositions over the parent drug. An example, without limitation, of a prodrug would be a derivative which is administered as an ester (the “prodrug”) to facilitate absorption in the gastrointestinal tract where improved water solubility is beneficial, but which then is metabolically hydrolyzed to a carboxylic acid and the active entity, Compound 1. A further example of a prodrug might be a short peptide (polyaminoacid) bonded to the hydroxyl group of Compound 1 wherein the peptide is metabolized to reveal the active moiety.

Prodrugs may be designed as reversible drug derivatives, for use as modifiers to enhance drug transport to site-specific tissues. The design of prodrugs to date has been to increase the effective water solubility of the therapeutic compound for targeting to regions where water is the principal solvent. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein in their entirety.

Additionally, prodrug derivatives of Compound 1 can be prepared by methods known to those of ordinary skill in the art (e.g., for further details see Saulnier et al., (1994), Bioorganic and Medicinal Chemistry Letters, Vol. 4, p. 1985). Prodrug forms of the herein described compounds, wherein the prodrug is metabolized in vivo to produce a derivative as set forth herein are included within the scope of the claims. Indeed, some of the herein-described compounds may be a prodrug for another derivative or active compound.

Sites on the aromatic ring portion of Compound 1 can be susceptible to various metabolic reactions, therefore incorporation of appropriate substituents on the aromatic ring structures, such as, by way of example only, halogens can reduce, minimize or eliminate this metabolic pathway.

Various methods of making Compound 1 are contemplated. In some embodiments, one or more of the following chemical reactions is performed in an inert atmosphere, for example, nitrogen or argon. In some embodiments, the temperature of the reaction is monitored. In some embodiments, the reaction is monitored by HPLC or TLC. In some embodiments, the pH of the reaction is monitored. In some embodiments, the temperature of the reaction is controlled. In some embodiments, the purity of the product is determined by HPLC. In some embodiments, the experiments are run on small scale, medium scale, large scale, analytical scale, or manufacturing scale. In some embodiments, the product is clarified by filtration through a pad comprising one or more of silica gel and celite.

In some embodiments, the synthesis is performed on large scale. In some embodiments, large scale comprises a scale of about 1 to about 10 kg. In some embodiments, the synthesis is performed on manufacturing scale. In some embodiments, manufacturing scale comprises a scale of greater than about 10 kg. In some embodiments, manufacturing scale comprises a scale of about 10 to about 1,000 kg. In some embodiments, manufacturing scale comprises a scale of about 10 to about 100 kg. In some embodiments, manufacturing scale comprises a scale of about 10 to about 50 kg. In some embodiments, manufacturing scale comprises a scale of about 33.4 kg.

In some embodiments, an experiment is performed on a smaller scale to gather information to be used to plan or perform synthesis on a manufacturing scale. In some embodiments, the results obtained on the smaller scales are expected to be reproducible on manufacturing scale. In some embodiments, the results obtained on smaller scales are not expected to be reproducible on manufacturing scale. In some embodiments, the yields obtained on manufacturing scale are greater than the yields obtained on smaller scales. In some embodiments, the yields obtained on manufacturing scale are lesser than the yields obtained on smaller scales.

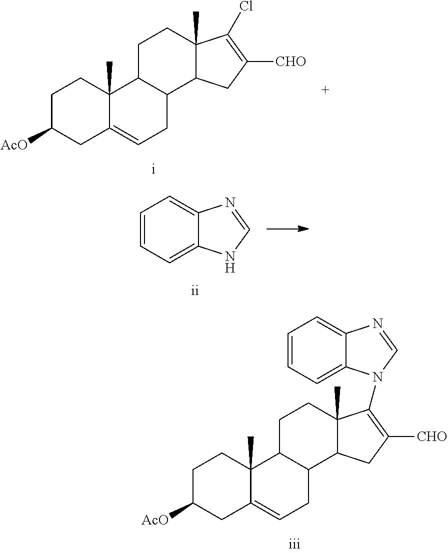 [SMILES: CC(=O)O[C@H]1CC[C@@]2(C)C(=CCC3C2CC[C@]2(C)C(Cl)=C(C=O)CC32)C1 ; c1ccc2[nH]cnc2c1 ; CC(=O)O[C@H]1CC[C@@]2(C)C(=CCC3C2CC[C@]2(C)C(n4cnc5ccccc54)=C(C=O)CC32)C1]

In one embodiment, a solution of a compound of Formula i in a solvent is prepared. A compound of Formula II is then contacted to the solution, and the resultant mixture is heated in the presence of a base for a period of time sufficient to provide a compound of Formula iii. In some embodiments, the period of time is about 1 hour, about 2 hours, about 4 hours, about 8 hours, about 12 hours, or about 24 hours. In some embodiments, the time is from about 1 hour to about 24 hours. In some embodiments, the base comprises lithium carbonate, sodium carbonate, potassium carbonate, sodium bicarbonate, a sodium phosphate, or a potassium phosphate. In some embodiments, the solvent comprises DMF. In some embodiments, the temperature is about 50° C., about 70° C., about 100° C., about 150° C., or a temperature effective to sustain reflux conditions. In some embodiments, the temperature is from about 50° C. to about 200° C. The compound of Formula iii can be isolated from the reaction mixture and purified by any method known to one of skill in the art. Such methods include, but are not limited to, pouring an aqueous mixture into the reaction mixture, thereby effecting the precipitation of compound iii as a solid. The isolated compound of Formula iii may optionally be purified by any method known to one of skill in the art. Such methods include, but are not limited to, trituration with water.

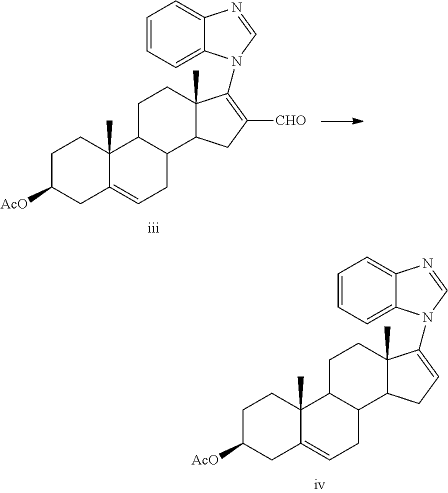 [SMILES: CC(=O)O[C@H]1CC[C@@]2(C)C(=CCC3C2CC[C@]2(C)C(n4cnc5ccccc54)=C(C=O)CC32)C1 ; CC(=O)O[C@H]1CC[C@@]2(C)C(=CCC3C2CC[C@]2(C)C(n4cnc5ccccc54)=CCC32)C1]

In one embodiment, a solution of a compound of Formula iii in a solvent is prepared, and the solution is contacted with a catalyst for a period of time sufficient to provide a compound of Formula iv. In some embodiments, the period of time is about 1 hour, about 2 hours, about 4 hours, about 8 hours, about 12 hours, or about 24 hours. In some embodiments, the time is from about 1 hour to about 24 hours. In some embodiments, the catalyst comprises palladium on carbon, platinum on carbon, a transition metal salt, or a transition metal complex. In some embodiments, the solvent comprises N-methylpyrrolidone. In some embodiments, the temperature is about 50° C., about 70° C., about 100° C., about 150° C., about 190° C., about 200° C. or a temperature effective to sustain reflux conditions. In some embodiments, the temperature is from about 50° C. to about 250° C. The compound of Formula iv can be isolated from the reaction mixture and purified by any method known to one of skill in the art. Such methods include, but are not limited to, in-line filtration. The isolated compound of Formula iii may optionally be purified by any method known to one of skill in the art.

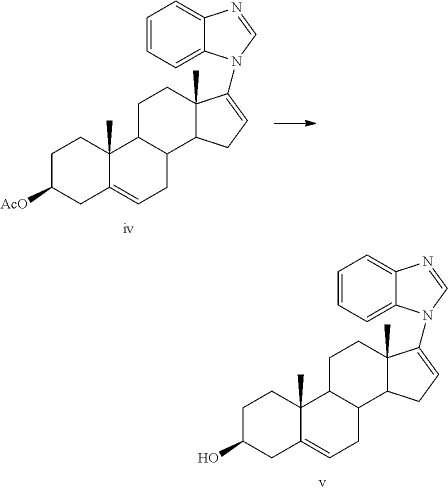 [SMILES: CC(=O)O[C@H]1CC[C@@]2(C)C(=CCC3C2CC[C@]2(C)C(n4cnc5ccccc54)=CCC32)C1 ; C[C@]12CC[C@H](O)CC1=CCC1C2CC[C@]2(C)C(n3cnc4ccccc43)=CCC12]

In one embodiment, a solution of a compound of Formula iv in a solvent is prepared, and the solution is contacted with a base for a period of time sufficient to provide a compound of Formula v (i.e., Compound 1). In some embodiments, the period of time is about 1 hour, about 2 hours, about 4 hours, about 8 hours, about 12 hours, or about 24 hours. In some embodiments, the time is from about 1 hour to about 24 hours. In some embodiments, the base comprises lithium hydroxide, sodium hydroxide, potassium hydroxide, sodium methoxide, potassium methoxide, sodium ethoxide, potassium ethoxide, lithium carbonate, sodium carbonate, potassium carbonate, sodium bicarbonate, a sodium phosphate, or a potassium phosphate. In some embodiments, the solvent comprises water, methanol, ethanol, 2-propanol, t-butanol, or mixtures thereof. In some embodiments, the solvent comprises methanol and the base comprises sodium methoxide. In some embodiments, the temperature is about 35° C., about 50° C., about 70° C., about 100° C., or a temperature effective to sustain reflux conditions. In some embodiments, the temperature is from about 25° C. to about 100° C. The compound of Formula v can be isolated from the reaction mixture and purified by any method known to one of skill in the art. Such methods include, but are not limited to, extraction. The isolated compound of Formula iii may optionally be purified by any method known to one of skill in the art. Such methods include, but are not limited to, trituration.

Pharmaceutical Composition/Formulation

A pharmaceutical composition, as used herein, refers to a mixture of a CYP17 inhibitor, such as, e.g., Compound 1 or other or 17-heteroarylsteroid, with other chemical components, such as carriers, stabilizers, diluents, dispersing agents, suspending agents, thickening agents, and/or excipients. In some embodiments, the composition can further comprise an additional therapeutic agent, such as a PI3K/Akt/mTOR inhibitor. In other embodiments of the invention, the additional therapeutic agent can be contained in a separate composition and administered concurrently or at different times from the composition comprising Compound 1 or another CYP17 inhibitor compound. The pharmaceutical composition facilitates administration of the compound and/or the additional therapeutic agent to an organism. Pharmaceutical compositions containing the CYP17 inhibitor compound or the additional therapeutic agent can be administered in therapeutically effective or sub-therapeutic amounts by any conventional form and route known in the art including, but not limited to: intravenous, oral, rectal, aerosol, parenteral, intramuscular, intradermal, subcutaneous, intraperitoneal, buccal, sublingual, mucosal, transcutaneous, ocular, ophthalmic, pulmonary, transdermal, vaginal, otic, nasal, and topical administration. The CYP17 inhibitor compound and the additional therapeutic agent can be administered using the same route or using different routes, for example via intravenous administration of the CYP17 inhibitor compound and oral administration of the additional therapeutic agent.

One may administer the compound in a local rather than systemic manner, for example, via injection of a composition of the invention directly into an organ, often in a depot or sustained release formulation. Furthermore, one may administer the pharmaceutical composition in a targeted drug delivery system, for example, in a liposome coated with organ-specific antibody. The liposomes will be targeted to and taken up selectively by the organ. In addition, the pharmaceutical composition may be provided in the form of a rapid release formulation, in the form of an extended release formulation, or in the form of an intermediate release formulation.

For oral administration, the CYP17 inhibitor compound and/or the additional therapeutic agent can be formulated readily by combining the active compounds with pharmaceutically acceptable carriers or excipients well known in the art. Such carriers enable the compounds described herein to be formulated as tablets, powders, pills, dragees, capsules, liquids, gels, syrups, elixirs, slurries, suspensions and the like, for oral ingestion by a patient to be treated.

Pharmaceutical preparations for oral use can be obtained by mixing one or more solid excipient with one or more of the compounds or agents described herein, optionally grinding the resulting mixture, and processing the mixture of granules, after adding suitable auxiliaries, if desired, to obtain tablets or dragee cores. Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations for oral use can also include polymers that are substantially insoluble in the acidic environment of the stomach, but are predominantly soluble in intestinal fluids at specific pHs. Such polymers are non-toxic, pharmaceutically acceptable polymers, and include, for example, cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., EUDRAGIT™ L30D55, EUDRAGIT™ FS30D, EUDRAGIT™ L100, KOLLICOAT™ EMM30D, ESTACRYL™ 30D, COATERIC™, and AQUATERIC™).

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. In certain instances, a gelatin is alkaline processed. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions may take the form of tablets, lozenges, or gels formulated in conventional manner. Parental injections may involve for bolus injection or continuous infusion. The pharmaceutical compositions of the invention may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

Compositions of the invention can be administered topically and can be formulated into a variety of topically administrable compositions, such as solutions, suspensions, emulsions, lotions, gels, pastes, medicated sticks, balms, creams, oils, or ointments. Such pharmaceutical compositions can contain solubilizers, stabilizers, tonicity enhancing agents, buffers and preservatives.

Formulations suitable for transdermal administration of compounds of the invention may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the CYP17 inhibitor compound and/or an additional therapeutic agent can be accomplished by means of iontophoretic patches and the like. Additionally, transdermal patches can provide controlled delivery of the CYP17 inhibitor compound or the additional therapeutic agent. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices can be in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

For administration by inhalation, the CYP17 inhibitor compound and/or the additional therapeutic agent may be in a form as an aerosol, a mist or a powder. Pharmaceutical compositions of the invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound and a suitable powder base such as lactose or starch.

The CYP17 inhibitor compound and/or the additional therapeutic agent may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In practicing the methods of treatment or use provided herein, therapeutically effective or sub-therapeutic amounts of a CYP17 inhibitor and an additional therapeutic agent as provided herein are administered in at least one pharmaceutical composition to a subject having a disease or condition to be treated. In some embodiments, the subject is a mammal, such as a human. A therapeutically effective amount can vary widely depending on the severity of the disease, the age and relative health of the subject, the potency of the compound used and other factors. The compounds can be used singly or in combination with one or more therapeutic agents as components of mixtures.

Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. Pharmaceutical compositions comprising a compound of the invention may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one pharmaceutically acceptable carrier, diluent or excipient and a compound of the invention described herein as an active ingredient in free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity.

In some embodiments, the pharmaceutical compositions are formulated such that the CYP17 inhibitor is substantially amorphous. By “amorphous”, it is meant that the majority of the compound in the composition is in an amorphous, e.g., non-crystalline form. In some embodiments, about 50% or more, about 55% or more, about 60% or more, about 65% or more, about 70% or more, about 75% or more, about 80% or more, about 85% or more, about 90% or more, about 95% or more of the compound is in a non-crystalline state. In particular embodiments, about 80% or more of the compound is in a non-crystalline state. In yet more particular embodiments, about 90% or more of the compound is in a non-crystalline state. In one embodiment, 95% or more of the compound is in a non-crystalline state. In some embodiments, the pharmaceutical compositions are formulated such that the CYP17 inhibitor is not substantially amorphous. Methods for determining whether a compound in a composition is amorphous are well known in the art, and include, but are not limited to Polarized Light Microscopy, X-Ray Powder Diffraction (XPRD), Electron Microscopy, Differential Scanning calorimetry (DSC), or other standard techniques.

In some embodiments, the CYP17 inhibitor is formulated as a solid dispersion composition. In some embodiments, the solid dispersion composition comprises the CYP17 inhibitor but not the additional therapeutic agent. In other embodiments, the solid dispersion composition comprises both the CYP17 inhibitor and the additional therapeutic agent.

In particular embodiments, the solid dispersion composition comprises the CYP17 inhibitor ad optionally the additional therapeutic agent, and a solid matrix. In more particular embodiments, the CYP17 inhibitor and optionally the additional therapeutic agent are dispersed in said matrix. In some embodiments, the solid dispersion of the compound in matrix is prepared by forming a homogeneous solution or melt of the CYP17 inhibitor and optionally the additional therapeutic agent, and a polymer, followed by solidifying the mixture, resulting in a solid composition of the CYP17 inhibitor dispersed in the solid matrix.

In some embodiments, the polymer is a water soluble polymer. Non-limiting examples of water soluble polymers used in solid dispersions include hydroxypropyl methyl cellulose (HPMC), hydroxypropyl cellulose (HPC), polyvinylpyrrolidone (PVP block copolymers of ethylene oxide and propylene oxide ((K-25, 50 30, 90; PVP), methyl cellulose (MC), and polyethyleneglycol (PEG). In other embodiments, the polymer is soluble in an aqeuous solution. In particular embodiments, the polymer is soluble in an aqueous solution which is pH 5.5 or greater. Non-limiting examples of polymers soluble in aqueous solutions of pH 5.5 or greater include sodium carboxymethylcellulose (NaCMC), sodium cellulose glycolate, and hydroxypropylmethyl cellulose acetate succinate (HPMCAS). Other non-limiting examples of polymers suitable for use in solid dispersions include, e.g., 3,4-dimethyl-phenomethylcarbamate (MPMC), hypromellose phthalate (HPMCP), Povidone K-90, poly(meth)acrylates (Eudragit), homopolymers of N-vinyl-2-pyrrolidone, povidone, copovidone (Plasdone), carboxymethylethylcellulose (CMEC), Poloxamer 188, Poloxamer 407, cellulose acetate phthalate (CAP), methacrylic copolymer LD (L30 D55), methacrylic copolymer S (S-100), aminoalkyl methacrylate copolymer E (gastric coating base), poly(vinyl acetal) diethylaminoacetate (AEA), ethylcellulose (EC), methacrylic copolymer RS (RS 30D), polyvinyl alcohol (PVA), HPMC 2208 (Metolose 90SH), HPMC 2906 (Metolose 65SH), HPMC (Metolose 60SH), hydroxypropylmethylcellulose (HPMC), dextrin, pullulan, Acacia, tragacanth, sodium alginate, propylene glycol alginate, agar powder, gelatin, starch, processed starch, phospholipids, lecithin, glucomannan, polyethyleneglycol (PEG) cellulose acetate trimellitate (CAT), hydroxypropyl methyl cellulose acetate trimellitate (HPMCAT), and carboxymethylcellulose acetate butyrate (CMCAB).

In some embodiments, preparation of the solid dispersion comprises forming a homogenous solution comprising the 17α-hydroxylase/C17,20-lyase inhibitor, the polymer, a solvent, and optionally the additional therapeutic agent, followed by solidifying the mixture by removal of the solvent. In some embodiments, the solvent is an organic solvent or a mixture of more than one organic solvent. Methods for removing the solvent from the mixture are known in the art, and can include freeze-drying, vacuum drying, spray-drying, or combinations thereof. In particular embodiments, the solid dispersion composition comprises both the CYP17 inhibitor and the additional therapeutic agent.

In particular embodiments, the solvent is removed by spray-drying. The term “spray-drying” generally refers to atomizing the solution into a spray of small droplets and rapidly evaporating the solvent from the droplets using a spray-drying apparatus. A description of spray-drying processes and spray-drying equipment can be found in Perry's Chemical Engineers' Handbook, pages 20-54 to 20-57 (Sixth Edition 1984). Solvent evaporation can be facilitated by, for example, maintaining the pressure in the spray-drying apparatus at a partial vacuum (for example, 0.01 to 0.50 atm), contacting the droplets with a warm drying gas, or a combination of these measures. In some embodiments, spray drying comprises contacting the spray of droplets with a drying gas.

In some embodiments, removal of the solvent by spray drying results in solid dispersion compositions in the form of particles. The particles can have a mean diameter of about 100 μm or less, about 95 μm or less, about 90 μm or less, about 85 μm or less, about 80 μm or less, about 75 μm or less, about 70 μm or less, about 65 μm or less, about 60 μm or less, about 55 μm or less, about 50 μm or less, about 45 μm or less, about 40 μm or less, about 35 μm or less, about 30 μm or less, about 25 μm or less, or about 20 μm or less. In some embodiments, the particles have a mean diameter of about 50-100 μm, about 30-75 μm, about 25-50 μm, about 20-30 μm, about 10-25 μm, or about 15-20 μm. Particle size can be measured using particle size measuring techniques known to those of skill in the art. Non-limiting examples of particle size measuring techniques include photon correlation spectroscopy, sedimentation field flow fractionation, laser diffraction or disk centrifugation. Another useful characteristic diameter of the droplets produced by an atomizer is D90, the droplet diameter corresponding to the diameter of droplets that make up 90% of the total liquid volume. In some embodiments, the particles of the composition have diameters spanning about 10-20 μm at D90, 15-20 μm at D90, or 17-19 μm at D90.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The compositions may be in liquid solutions or suspensions, solid forms suitable for solution or suspension in a liquid prior to use, or as emulsions. These compositions may also contain minor amounts of nontoxic, auxiliary substances, such as wetting or emulsifying agents, pH buffering agents, and so forth.

A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

### EXAMPLES

### Example 1

### In Vitro Analysis of Combination Treatment

PC3 (CRL-1435) and LNCaP (CRL-1740) cells will be maintained in RMPI media supplement with 10% heat inactivated fetal bovine serum, 2 mM L-glutamine, 100 U/ml penicillin G sodium/100 mg/ml streptomycin sulfate, sodium pyruvate, and non-essential amino acids at 37° C. in a humidified 5% CO2 incubator. LAPC-4 cells will be maintained similarly, but in IMDM media supplemented with 5% heat inactivated fetal bovine serum. Cells expressing either the wild type (WT) or AR mutant proteins were created by stable transfection of PC3 (AR null) cells with pCIneo-hAR (WT), pCIneo-hAR-W741C, or pCIneo-hAR-W741L. Cells can be cultured in phenol red-free, steroid-free media, consisting of basal media supplemented with 5-10% dextran-coated, charcoal-stripped FBS. Compound 1 will be prepared as described and dissolved in DMSO prior to use. The PI3K/Akt/mTOR inhibitor will be dissolved in a suitable solvent, such as water, ethanol or DMSO, prior to use. Cells will be treated with one, both, or neither Compound 1 and the PI3K/Akt/mTOR inhibitor. Protein expression, proliferation, and survival will be measured as described in the examples below.

### Example 2

### Immunoblot and Protein Analysis

Whole cell extracts can be prepared by collecting cells from in vitro cultures or from biological samples taken from a test subject, washing the cell pellet with 1× cold PBS, extracting with lysis buffer at 4° C. for 1 hour followed by the removal of cell debris by centrifugation at 14,000×g for 20 min at 4° C. Protein concentrations can be determined using the Bio-Rad protein assay system (Bio-Rad Laboratories, Richmond, Calif.). Equal amounts of protein can be resolved by SDS-PAGE, transferred to PVDF membrane and stained with SYPRO Ruby. Membranes can then be blocked for 1 hr at room temperature or 4° C. overnight with 5% non-fat dry in TBS-T (10 mM Tris, pH 7.4+0.05% Tween-20). After treatment with the appropriate primary and secondary antibodies in 5% milk in TBS-T, enhanced chemiluminescence is performed (Amersham, Piscataway, N.J.). The following antibodies (clone, dilution) can be used to detect relevant proteins: anti-androgen receptor (clone F39.4.1, BioGenenix, San Ramon, Calif., 1:400), anti-α-tubulin (clone B-5-1-2, Sigma, St. Louis, Mo.; 1:3000), anti-β-actin (clone AC-15, Abcam, Cambridge, Mass., 1:5000), anti-PARP (clone F-2, Santa Cruz Biotechnology, 1:200), anti-pro-caspase-3 (clone E-8, Santa Cruz Biotechnology 1:200), anti-Bcl-xL (clone H-62, Santa Cruz Biotechnology, 1:200), anti-PSA (clone A67-B/E3) and anti-AR (Millipore, Temecula, Calif., 1:500), anti-XIAP (clone 2F-1, Abcam, Cambridge, Mass., 1:1000), anti-total eIF4E, eIF4G, or 4EBP1 and phospho-4EBP1 (Cell Signaling Technology, Danvers, Mass.). Quantitation of protein expression will be determined using Image J analysis.

### Example 3

### Isolation of RNA and qRT-PCR

Total RNA can be isolated from cellular samples using QIAGEN's RNeasy kit (Qiagen, Valencia, Calif.) and quantified using a Nanodrop. cDNA is primed using random hexamers and the Superscript II RT enzyme (Invitrogen, Carlsbad, Calif.) according to the manufacturer's directions. The PCR step is performed using the EvaGreen-R qPCR supermix (ABM, BC, Canada) according to the manufacturer's instructions. qPCR reactions are performed using an ABI 7900 real time PCR system with the following cycling conditions: 50° C., 2 minutes, 1×; 95° C., 10 minutes, 1×; 94° C., 20 s, 60° C., 1 minute, 40×. A dissociation step can also be performed to confirm amplification of a single product. The relative standard curve method is used to quantify the amount of AR and RPLPO mRNA in each sample. A cDNA standard curve of serial dilutions will be obtained using cDNA from DMSO-treated cells for amplification with both AR and RPLPO primers. Relative gene expression was determined by using the relative standard curve method.

### Example 4

### Apoptosis Analysis by Annexin V Staining

Cells can be obtained from test subjects treated with control, Compound 1 alone, an mTOR inhibitor (e.g. everolimus) alone, or both Compound 1 and the mTOR inhibitor for varying time periods, such as ranging from 1 day to 90 or more days. Apoptotic cells can be measured with the Annexin V-FITC Apoptosis Detection Kit (BD Biosciences, San Jose, Calif.). Propidium iodide (1 mg/mL) is added just prior to flow cytometric analysis (Becton Dickinson FACScan). Ten thousand cells per sample can be analyzed, and a percentage of apoptotic cells calculated. Apoptosis studies will be repeated a minimum of two times.

### Example 5

### Luciferase Assays and Cell Proliferation Assays—Determination of AR Transcriptional Activity

The plasmid pARE4-luciferase contains four Androgen Response Elements (AREs) cloned in tandem into pGL3 (Promega, Madison, Wis.). pRL-CMV-Renilla is a cytomegalovirus (CMV) promoter-driven Renilla luciferase control plasmid. PC3 cells stably expressing WT or mutant AR proteins are seeded into poly-lysine-coated plates using phenol red-free, steroid-free RMPI complete media without antibiotics and transfected 24 h later with 100 ng pARE4-Luciferase and 100 pg pRL-CMV-Renilla using Lipofectamine 2000 (Invitrogen, Carlsbad, Calif.). 24 h post-transfection, the medium is changed to fresh phenol red-free, steroid-free RMPI complete media and control or combination treatment is added at varying concentrations. Firefly and Renilla luciferase activities are determined 18 h later using the Dual Luciferase Kit (Promega, Madison, Wis.). Mean and standard deviations will be calculated based on at least three independent experiments performed in triplicate. To determine IC₅₀ values of Compound 1 in the presence or absence of a PI3K/Akt/mTOR inhibitor, dose-response data will be analyzed by non-linear regression to fit the data to the log (Compound 1) vs. response with variable slope using Graphpad Prism software. IC50 values of the PI3K/Akt/mTOR inhibitor can also be determined in the presence or absence of Compound 1.

### Example 6

### Treating Prostate Cancer Using Combination Therapy of Compound 1 and Everolimus

Combination treatment using Compound 1 and everolimus will be compared to treatment using everolimus alone. The trial population will include males aged 18 years or more who have confirmed adenocarcinoma of the prostate and progressing disease despite androgen ablation therapy. Progressing disease can be defined as having prostate specific antigen (PSA) levels that have risen on at least two successive occasions at least 1 week apart, wherein the most recent PSA level ≧4 ng/mL. Subjects will be randomly divided into a trial (Compound 1 and everolimus) and a control (everolimus alone) group.

Subjects will take an oral composition containing the trial drug (e.g., a composition containing both Compound 1 and everolimus) once daily, for 12 weeks. Screening and other testing will occur once every 2 weeks. Treatment may continue until disease progression, subject withdrawal, unacceptable toxicity, or at the Investigator's discretion. The trial group can be further divided into subgroups to be treated with different dosages. Suitable dosages can be based on prior safety data obtained on treating with Compound 1 and/or with everolimus, animal model trials, and an initial safety dosage escalation test. For example, dosages can comprise 5 mg or 10 mg of everolimus with 650 mg, 1300 mg, or 1950 mg of Compound 1.

Screening of tumor response can be determined using Response Evaluation Criteria in Solid Tumors (RECIST) criteria using X-ray, CT, and magnetic resonance screening (MRI). The technique is recommended for National Cancer Institute (NCI)-sponsored trials and involves formalized rules for measurement of tumor target lesions. RECIST criteria are a voluntary, international standard, and are not an NCI standard. They are based on a simplification of former methods [World Health Organization (WHO), ECOG] and based on measurable disease, (i.e., the presence of at least one measurable lesion). RECIST criteria offer a simplified, conservative, extraction of imaging data for wide application in clinical trials. RECIST (Eisenhauer et al., 2009, incorporated by reference herein) is used in this trial. Tumor biopsies can also be obtained at specified time points or at the conclusion of trial treatment to further follow effects of treatment on cancer progression. Other characteristics to be collected during the trial include PSA levels, survival data, such as time to progression (TTP), progression-free survival (PFS), and overall survival (OS).

Time-to-progression (TTP) is defined as the time from first dose of Trial Drug to first documented PI evaluation of the disease becoming worse, based on clinical course, radiological evidence, and biochemical markers (PSA) results.

Progression-free survival (PFS) is the length of time during and after treatment in the trial in which a subject is living with the disease (CRPC) that does not worsen.

Overall Survival (OS) is defined as the time from first dose of Trial Drug to first documentation of death due to any cause. For the purpose of this trial OS is reported as percentage of survived subjects 5 years after receiving the first dose of Trial Drug.

The analysis of TTP, PFS, and OS will use log-rank tests for the comparison between trial and control groups. Kaplan-Meier estimates will be plotted by treatment group. Median time to events with a 95% confidence interval, if estimable, will also be tabulated by treatment group.

### Example 7

### Single Agent Growth Inhibitory Activity and Combination Growth Inhibitory Activity Determination

The in vitro IC₅₀ of the growth inhibitory activity of 12 test agents against 5 human tumor cell lines will be determined, as well as the combination effects of galeterone (TOK-001) with 6 test agents. The IC50 of the single agents and combination effects will be tested in each of the 5 cell lines using the Chou-Talalay combination analysis method. Cell growth will be determined using Promega's Cell Titer-Glo® assay.

The antiproliferative activity of the study test agents (see below) against the following panel of human tumor cell lines will be determined with Promega's Cell Titer-Glo® assay:

| Tumor Type | Cell Line | Test Agent |
|---|---|---|
| Prostate | PC3 | Galeterone |
| | LNCaP | Abiraterone |
| | VCaP | Everolimus |
| | C4-2 | AZD8055 |
| | MCF-7 | BEZ235 |
| | | XL765 |
| | | Dasatinib |
| | | AZD5363 |
| | | MDV-3100 |
| | | Casodex |
| | | Ketoconazole |
| Breast | | TAK-700 |

The human tumor cells will be grown according to standard conditions and standard medium during routine growth and passage. Three days prior to plating cells for the IC₅₀ experiment, the cells will be transferred into Phenol-red free medium containing charcoal/dextran treated FBS. **DHT will be added to the medium at a final concentration of 1 nM** TBD

On what will be considered as Day −1, the cells will be plated into 96-well microculture plate (Costar white, flat bottom #3917) in a total volume of 90 μL/well. An additional plate will be included to serve as a Time 0 reference for cell growth. After 24 hours of incubation in a humidified incubator at 37° C. with 5% CO₂ and 95% air, 10 μL of 10×, serially diluted test agents in growth medium will be added to each well. This will be considered Day 0. The TO plate will be developed using CellTiter Glo for a reference cell number. On Day 3 and Day 6, the medium will be removed and replaced with fresh media/drugs. On Day 7, after 192 total hours of culture in a CO₂ incubator (168 total hours of drug treatment), the plated cells and Cell Titer-Glo® (Promega # G7571) reagents will be brought to room temperature to equilibrate for 30 minutes. 100 μL of Cell Titer-Glo® reagent will be added to each well. The plate will be shaken for 2 minutes and then left to equilibrate for 10 minutes before reading luminescence on the Tecan GENios microplate reader.

Percent inhibition of cell growth will be calculated relative to untreated control wells. All tests will be performed in duplicate at each concentration level.

The IC₅₀ value for the test agents will be estimated using Prism 3.03 by curve-fitting the data using the following four parameter-logistic equation:

$$Y = \frac{Top - Bottom}{1 + {( \frac{X}{{IC}_{50}} )}^{n}} + Bottom$$

where Top is the maximal % of control absorbance, Bottom is the minimal % of control absorbance at the highest agent concentration, Y is the % of control absorbance, X is the agent concentration, IC₅₀ is the concentration of agent that inhibits cell growth by 50% compared to the control cells, and n is the slope of the curve.

Galeterone will be tested in combination with 6 different test agents: Everolimus, AZD8055, BEZ235, XL765, Dasatinib and AZD5363 using the constant ratio combination design of Chou and Talalay. The same Promega CellTiter Glo® assay described above (time course, medium composition and drug treatment) will be used in the combination study.

The individual IC₅₀ values of the test agents determined as described above will be used to determine appropriate drug ratios and concentration ranges for a combination study based on the constant ratio design of Chou-Talalay. Assuming a combination response of near additivity, drug concentrations will bracket the sum of one half of the respective IC₅₀'s with serial dilutions selected based upon the inhibition curves of the agents being combined, (typically 1.5 fold dilutions) with a total of 7 drug concentrations. Each concentration will be tested in quadruplicate.

Data Treatment.

CalcuSyn Software developed by T.-C. Chou (distributed by BioSoft) will be used to determine whether the Dynamix test agent and the selected anticancer agents produce synergistic, additive, or antagonistic effects.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for the treatment of a cancer in a human subject, the method comprising:
administering to a subject having a cancer Compound (I): 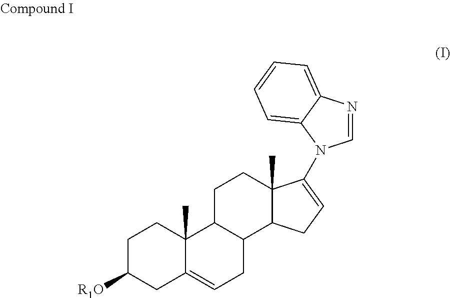
or a pharmaceutically acceptable salt, analog, N-oxide, prodrug, or solvate thereof, wherein: R₁ is H or acetyl,
and an additional therapeutic agent, wherein the additional therapeutic agent is a PI3K inhibitor and/or mTOR inhibitor.

2. A method for the treatment of a cancer in a human subject, the method comprising:
administering to a subject having a cancer abiraterone alcohol or abiraterone acetate, or a pharmaceutically acceptable salt, analog, N-oxide, prodrug, or solvate thereof, and an additional therapeutic agent, wherein the additional therapeutic agent is a PI3K inhibitor and/or mTOR inhibitor.

3. A method of treating prostate cancer in a human subject in need thereof, comprising:
administering a 17α-hydroxylase/C17,20-lyase inhibitor (CYP17 inhibitor) and at least one additional therapeutic agent, wherein the additional therapeutic agent is a PI3K inhibitor and/or mTOR inhibitor, to said subject if blood PSA levels in said subject has increased in at least two successive occasions at least one week apart.

4. A method of treating prostate cancer in a human subject in need thereof, comprising:
administering a CYP17 inhibitor and at least one additional therapeutic agent, wherein the additional therapeutic agent is a PI3K inhibitor and/or mTOR inhibitor, to said subject if blood PSA levels is 4 ng/ml or above.

5. A method of treating prostate cancer in a human subject in need thereof, comprising:
administering a CYP17 inhibitor and at least one additional therapeutic agent, wherein the additional therapeutic agent is a PI3K inhibitor and/or mTOR inhibitor, if said subject is determined to harbor a mutation or copy number variation in a gene associated with the PI3K/mTOR pathway.

6. The method of claim 5, wherein said mutation or copy number variation is selected from the group consisting of PTEN mutations, PTEN loss-of-heterozygosity, PIK3CA mutations, PIK3CA amplifications, AKT mutations, AKT amplifications, and P85α mutations.

7. The method of any of claims 3-5, wherein said CYP17 inhibitor is Compound I, abiraterone alcohol, or abiraterone acetate.

8. The method of any of the preceding claims, wherein the mTOR inhibitor directly binds and inhibits mTORC1 and mTORC2.

9. The method of any of the preceding claims, wherein the mTOR inhibitor is selectively active against mTORC1 as compared to mTORC2.

10. The method of any of the preceding claims, wherein the mTOR inhibitor is rapamycin, temsirolimus, umirolimus, zotarolimus, or any analogues or derivatives thereof.

11. The method of any of the preceding claims, wherein the mTOR inhibitor is not everolimus.

12. The method of any of the preceding claims, wherein the mTOR inhibitor is not rapamycin or a rapamycin analog.

13. The method of any of the preceding claims, wherein the mTOR inhibitor also inhibits PI3K.

14. The method of any of the preceding claims, wherein the mTOR inhibitor is a TOR kinase inhibitor (TOR-KI).

15. The method of any of the preceding claims, wherein the mTOR inhibitor is OSI-027, INK-128, AZD-8055, AZD-2014, Palomid 529, Pp-242, BEZ235, AZD-8055, BGT226, XL765, GDC-0980, GSK2126458, PF-04691502, PF-05212384, or any analogues or derivatives thereof.

16. The method of any of the preceding claims, wherein the PI3K inhibitor is a pan-PI3K inhibitor.

17. The method of any of the preceding claims, wherein the PI3K inhibitor selectively inhibits a class I PI3K family member relative to at least one other class I PI3K family member.

18. The method of any of the preceding claims, wherein the PI3K inhibitor selectively inhibits PI3Kα, PI3Kβ, PI3Kγ, PI3Kδ, or some combination thereof.

19. The method of any of the preceding claims, wherein the PI3K inhibitor also inhibits mTOR.

20. The method of any of the preceding claims, wherein the PI3K inhibitor is SF1126, SF1101, BEZ235, BKM120, BYL719, BGT-226, XL-147, GDC-0941, ZSTK-474, PX-866, GDC-0980, PKI-587, PF-04691502, BWT33597, PI-103, CAL-101, GNE-477 or any derivatives thereof.

21. The method of any of the preceding claims, wherein R1 is H.

22. The method of any of the preceding claims, wherein the subject is a human.

23. The method of any of the preceding claims, wherein the cancer comprises a heterogeneous tumor.

24. The method of any of the preceding claims, wherein Compound (I) or abiraterone alcohol or abiraterone acetate and the additional therapeutic agent are administered concurrently to the subject.

25. The method of any of the preceding claims, wherein Compound (I) or abiraterone alcohol or abiraterone acetate and the additional therapeutic agent are administered separately to the subject.

26. The method of any of the preceding claims, wherein the cancer is bone cancer, breast cancer, cervical cancer, endometrial cancer, leukemia, lung cancer, lymphoma, ovarian cancer, prostate cancer, skin cancer, or testicular cancer.

27. The method of claim 26, wherein the cancer is prostate cancer or breast cancer.

28. The method of claim 27, wherein the prostate cancer is castration-resistant prostate cancer.

29. The method of any of the preceding claims, comprising administering Compound (I), abiraterone alcohol, or abiraterone acetate and the additional therapeutic agent for a period of about 3 days to about 12 months.

30. The method of any of the preceding claims, comprising administering Compound (I), abiraterone alcohol, or abiraterone acetate and the additional therapeutic agent for a period of about 28 days to about 3 months.

31. The method of any of the preceding claims, comprising administering Compound (I), abiraterone alcohol, or abiraterone acetate and the additional therapeutic agent for a period of over 45 days.

32. The method of any of the preceding claims, comprising administering Compound (I), abiraterone alcohol, or abiraterone acetate and the additional therapeutic agent for a period of over 60 days.

33. The method of any of the preceding claims, comprising administering Compound (I), abiraterone alcohol, or abiraterone acetate and the additional therapeutic agent for a period of over 90 days.

34. The method of any of the preceding claims, comprising administering between about 30 to about 175 mg/kg/day of Compound (I), abiraterone alcohol, or abiraterone acetate.

35. The method of any of the preceding claims, comprising administering between about 25 mg/kg/day to about 50 mg/kg/day of Compound (I), abiraterone alcohol, or abiraterone acetate.

36. The method of any of the preceding claims, comprising administering less than 50 mg/kg/day of Compound (I), abiraterone alcohol, or abiraterone acetate.

37. The method of any of the preceding claims, comprising administering about 325 mg to about 3500 mg of Compound (I), abiraterone alcohol, or abiraterone acetate.

38. The method of any of the preceding claims, comprising administering between 900 mg and 1950 mg of Compound (I), abiraterone alcohol, or abiraterone acetate.

39. The method of any of the preceding claims, comprising administering about 650 mg, about 975 mg, about 1300 mg, or about 1950 mg of Compound (I), abiraterone alcohol, or abiraterone acetate.

40. The method of any of the preceding claims, comprising administering between about 0.01 and 10 mg/kg of the additional therapeutic agent.

41. The method of claim 40, comprising administering between about 0.01 and 1 mg/kg of the additional therapeutic agent.

42. The method of claim 40, comprising administering between about 0.1 and 2 mg/kg of the additional therapeutic agent.

43. The method of claim 40, comprising administering between about 0.5 and 5 mg/kg of the additional therapeutic agent.

44. The method of claim 40, comprising administering between about 1 and 10 mg/kg of the additional therapeutic agent.

45. The method of any of the preceding claims, where the cancer tumor volume decreases after the administration of Compound (I), abiraterone alcohol, or abiraterone acetate and the additional therapeutic agent for said period.

46. The method of any of the preceding claims, where the cancer tumor volume remains stable after the administration of Compound (I), abiraterone alcohol, or abiraterone acetate and the additional therapeutic agent for said period.

47. The method of any of the preceding claims, where the cancer remains stable as characterized by RECIST guidelines during administration of Compound (I), abiraterone alcohol, or abiraterone acetate and the additional therapeutic agent for said period.

48. The method of any of the preceding claims, comprising administering Compound (I), abiraterone alcohol, or abiraterone acetate and/or the additional therapeutic agent to a subject one, two, three, four, five, six, seven, eight, nine, or ten times per day.

49. The method of any of the preceding claims, comprising administering parenterally, intravenously, intramuscularly, intradermally, subcutaneously, intraperitoneally, orally, buccally, sublingually, mucosally, rectally, transcutaneously, transdermally, ocularly, or by inhalation.

50. The method of any of the preceding claims, wherein Compound (I), abiraterone alcohol, or abiraterone acetate is administered as a tablet, a capsule, a cream, a lotion, an oil, an ointment, a gel, a paste, a powder, a suspension, an emulsion, or a solution.

51. The method of any of the preceding claims, wherein Compound (I), abiraterone alcohol, or abiraterone acetate is formulated as a solid dispersion composition.

52. The method of claim 51, wherein said solid dispersion composition is a spray dried dispersion composition.

53. The method of any of the preceding claims, comprising administering a therapeutically effective amount of Compound (I), abiraterone alcohol, or abiraterone acetate.

54. The method of any of the preceding claims, comprising administering a therapeutically effective amount of the additional therapeutic agent.

55. The method of any of the preceding claims, wherein a sub-therapeutic amount of Compound (I), abiraterone alcohol, or abiraterone acetate is administered.

56. The method of any of the preceding claims, wherein a sub-therapeutic amount of the additional therapeutic agent is administered.

57. The method of any of the preceding claims, wherein said administration of Compound (I), abiraterone alcohol, or abiraterone acetate and the additional therapeutic agent results in a synergistic effect, wherein the synergistic effect is evidenced by a therapeutic effect of administering both Compound (I) and the additional therapeutic agent to a test subject that is more than the additive effects of administering only Compound (I) to a test subject and administering only the additional therapeutic agent to a test subject.

58. The method of any of the preceding claims, wherein the additional therapeutic agent inhibits a PI3K or mTOR complex with a potency of less than 1 μM in an in vitro assay.

59. The method of any of the preceding claims, wherein the additional therapeutic agent inhibits a PI3K or mTOR complex with a potency of less than 500 nM in an in vitro assay.

60. The method of any of the preceding claims, wherein the additional therapeutic agent inhibits a PI3K or mTOR complex with a potency of less than 100 nM in an in vitro assay.

61. A pharmaceutical composition comprising a CYP17 inhibitor and at least one additional therapeutic agent, wherein the additional therapeutic agent is a PI3K inhibitor and/or mTOR inhibitor.

62. The pharmaceutical composition of claim 61, wherein the CYP17 inhibitor is a compound of Formula (II): 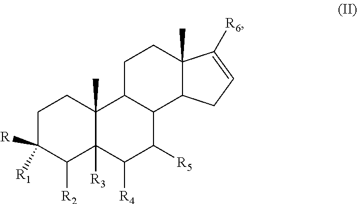
wherein:
either R and R1 are independently H, OH, SH, NH2, N(R7), NHR7, F, OR7, or O(C═O)R7; or R and R1 together form a ketone or an exo-methylene;
a. each occurrence of R₇ is independently H, C₁-C₈-alkyl, arakyl, alkylaryl, alkoxyalkyl, aryl, 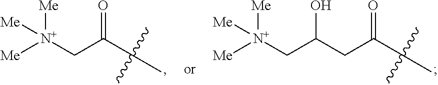 [SMILES: *C(=O)C[N+](C)(C)C ; *C(=O)CC(O)C[N+](C)(C)C]
b. R₂, R₃, R₄, and R₅ are independently H, OH, SH, NH₂, or NHR₇, or together with a neighboring R₂, R₃, R₄, or R₅ form an olefinic bond;
c. R₆ is: a 1-azaazulen-3-yl; 2-alkylindazol-3-yl; pyrazolo-[1,5-a]-pyridin-3-yl; imidazo-[1,2-a]-pyridin-3-yl; pyrazolo-[2,3-a]-pyrimidin-3-yl; pyrazolo-[2,3-c]-pyrimidin-3-yl; imidazo-[1,2-c]-pyrimidin-3-yl; imidazo-[1,2-a]-pyrimidin-3-yl; 4-alkylpyrazolo-[1,5-a]imidazol-3-yl; 2,1-benzoxazol-3-yl; 2,1-benzthiazol-3-yl; imidazo[2,1-b][1,3]oxazol-5-yl; imidazo[2,1-b][1,3]thiazol-5-yl; imidazo-[2,1-b][1,2]isoxazol-6-yl; or 1,2-benzisoxazol-3-yl, group, wherein any of the foregoing groups are optionally-substituted; or a bicyclic structure of Formula III: 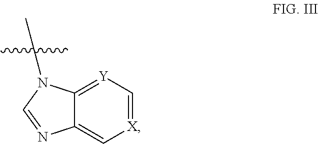
wherein X and Y are independently CH or N, and the bicyclic structure of Formula III is optionally substituted with halogen, chalcogen or C₁-C₄-alkyl; or
wherein R₆ is a bicyclic structure of Formula III wherein one of X and Y is N and the other of X and Y is CH when one or both of R and R₁ are 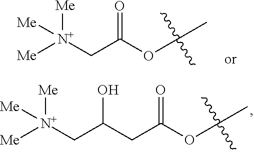 [SMILES: *OC(=O)C[N+](C)(C)C ; *OC(=O)CC(O)C[N+](C)(C)C]
or an analog, a derivative, a metabolite or a pharmaceutically-acceptable salt of any of the foregoing.

63. The pharmaceutical composition of claim 62, wherein R6 is an unsubstituted benzimidazole or amidazole.

64. The pharmaceutical composition of claim 63, wherein R6 is an unsubstituted benzimidazaole.

65. The pharmaceutical composition of claim 62, wherein the compound is Compound I or abiraterone alcohol or abiraterone acetate: 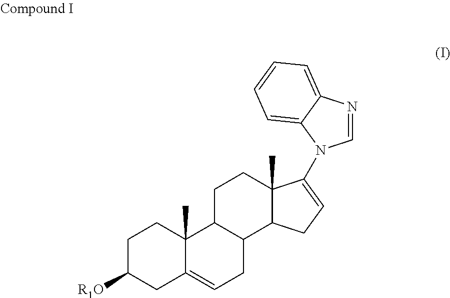
or a pharmaceutically acceptable salt, analog, N-oxide, prodrug, or solvate thereof, wherein: R₁ is H or acetyl.

66. The pharmaceutical composition of any of the preceding claims, comprising about 50 to about 3500 mg of said CYP17 inhibitor.

67. The pharmaceutical composition of any of the preceding claims, comprising about 50 to about 3500 mg of said CYP17 inhibitor and about 5 to about 500 mg of said PI3K inhibitor or mTOR inhibitor.

68. The pharmaceutical composition of any of the preceding claims, wherein said mTOR inhibitor directly binds to and inhibits both mTORC1 and mTORC2.

69. The pharmaceutical composition of any of the preceding claims, wherein said mTOR inhibitor selectively inhibits mTORC1 as compared to mTORC2.

70. The composition of any of the preceding claims, wherein the mTOR inhibitor is rapamycin, temsirolimus, umirolimus, zotarolimus, or any analogues or derivatives thereof.

71. The composition of any of the preceding claims, wherein the mTOR inhibitor is not everolimus.

72. The composition of any of the preceding claims, wherein the mTOR inhibitor is not rapamycin or a rapamycin analog.

73. The composition of any of the preceding claims, wherein the mTOR inhibitor also inhibits PI3K. The composition of any of the preceding claims, wherein the mTOR inhibitor is a TOR kinase inhibitor (TOR-KI).

74. The composition of any of the preceding claims, wherein the mTOR inhibitor is OSI-027, INK-128, AZD-8055, AZD-2014, Palomid 529, Pp-242, BEZ235, AZD-8055, BGT226, XL765, GDC-0980, GSK2126458, PF-04691502, PF-05212384, or any analogues or derivatives thereof.

75. The composition of any of the preceding claims, wherein the PI3K inhibitor is a pan-PI3K inhibitor.

76. The composition of any of the preceding claims, wherein the PI3K inhibitor selectively inhibits a class I PI3K family member relative to at least one other class I PI3K family member.

77. The composition of any of the preceding claims, wherein the PI3K inhibitor selectively inhibits PI3Kα, PI3Kβ, PI3Kγ, PI3Kδ, or some combination thereof.

78. The composition of any of the preceding claims, wherein the PI3K inhibitor also inhibits mTOR.

79. The composition of any of the preceding claims, wherein the PI3K inhibitor is SF1126, SF1101, BEZ235, BKM120, BYL719, BGT-226, XL-147, GDC-0941, ZSTK-474, PX-866, GDC-0980, PKI-587, PF-04691502, BWT33597, PI-103, CAL-101, GNE-477 or any derivatives thereof.

80. The composition of any of the preceding claims, wherein said composition is formulated as a pill, a tablet or a capsule.

81. The composition of any of the preceding claims, wherein said composition is formulated as a syrup, emulsion, or suspension.

82. The composition of any of the preceding claims, wherein said composition is formulated as a solid dispersion.

83. The composition of claim 82, wherein said solid dispersion is a spray dried dispersion.
